(19) United States
(12) Patent Application Publication
(10) Pub. No.: US 2025/0249077 A1
(43) Pub. Date: Aug. 7, 2025

(54) NAMPT FOR WOUND-HEALING AND STIMULATING HAIR GROWTH AND/OR REGROWTH

(71) Applicant: Innovative Surgical Regeneration Holdings, LLC, Mandeville, LA (US)

(72) Inventors: Jay RAPPAPORT, Covington, LA (US); Amin IZADPANAH, Covington, LA (US); Reza IZADPANAH, Covington, LA (US)

(21) Appl. No.: 18/854,782

(22) PCT Filed: Apr. 10, 2023
(86) PCT No.: PCT/US2023/065582

§ 371 Date: Oct. 7, 2024

Related U.S. Application Data
(60) Provisional application No. 63/329,224, filed on Apr. 8, 2022.
(60) Provisional application No. 63/481,962, filed on Jan. 27, 2023.
(60) Provisional application No. 63/481,963, filed on Jan. 27, 2023.

(51) Int. Cl. A61K 38/45; A61K 9/00; A61K 9/06; A61K 45/06; A61P 17/02; A61P 17/14

(52) U.S. Cl. CPC A61K 38/45; A61K 9/0014; A61K 9/06; A61K 45/06; A61P 17/02; A61P 17/14

(57) ABSTRACT

A method for stimulating hair growth and/or regrowth in a subject is described. The method includes contacting a target region of skin of the subject with an effective amount of a hair growth or regrowth composition comprising a nicotinamide phosphorylribosyltransferase (NAMPT) protein, an active peptide fragment thereof, a NAMPT analog, a vector comprising polynucleotide encoding a NAMPT protein, a NAMPT activator, or a combination thereof.

### PRIOR RELATED APPLICATIONS

This invention claims priority to U.S. 63/481,962, filed Jan. 27, 2023, U.S. 63/481,963, filed Jan. 27, 2023, and 63/329,224, filed Apr. 8, 2022, each of which is incorporated by reference in its entirety herein for all purposes.

### FEDERALLY SPONSORED RESEARCH STATEMENT

This invention was made with government support under Grant No. 2P510DO11104-57 awarded by the National Institutes of Health. The government has certain rights in the invention.

### FIELD OF THE DISCLOSURE

The present disclosure relates to effective therapies at point of care to promote rapid and regenerative healing outcomes. Specifically provided are local, topical application of NAMPT in combination with NMN and NR using Pluronic-F127 hydrogel or fibrin hydrogel to accelerate wound healing by promoting epithelialization, rather than scarring and contraction, in wound models through increased NAD biosynthesis and growth factor signaling.

The present disclosure also relates to effective therapies to promote growth and regeneration of hair follicles. Specifically provided is application of NAMPT to accelerate hair growth and promote regeneration of hair follicles.

### BACKGROUND OF THE DISCLOSURE

Skin is the largest organ in the human body and consists of several tissues and cell types: the epidermis, dermis, hair follicles, various glands such as sebaceous glands, vasculature, and stem cells. Skin damage results in non-functional scar tissue, characterized by lack of normal skin architecture, and replaced by fibroblasts. Despite advancements that enhance “wound closure and healing”, there is great need for therapies that fully regenerate the skin to the normal state.

Additionally, hair loss is of concern to a large number of men and women. In many individuals, hair loss (i.e., alopecia) causes embarrassment, and/or psychological problems such as depression. Although alopecia is more common in men (e.g., male pattern baldness or androgenic alopecia) than women (e.g., female pattern baldness), it is a significant concern to both men and women. Methods of treating hair loss include administration of medications such as minoxidil, finasteride, and dutasteride, or hair transplantation.

The hair follicle is a structure located within the skin that is responsible for hair production. The hair follicle consists of various cell types, including stem cells and progenitor cells, which contribute to the production of hair as well as skin repair and regeneration during skin injury. Promotion and enhancement of hair follicles is one of the major goals of regenerative skin therapies.

Nicotinamide adenine dinucleotide (NAD) is essential to cellular metabolism. NAD insufficiency leads to impaired cellular energy production. Nicotinamide phosphoribosyltransferase (NAMPT) is the rate-limiting biosynthetic enzyme in the salvage NAD synthesis pathway and exists intracellularly and extracellularly. Extracellular NAMPT functions as a growth factor and cytokine and exhibits NAD biosynthetic activity.

### SUMMARY OF THE DISCLOSURE

The present invention provides for the use of NAMPT and related compounds to improve wound healing and/or stimulate hair growth and promote the growth and/or regeneration of hair follicles.

The present disclosure combines an NAD-producing enzyme, that dually acts as an enzyme and growth factor/cytokine, with NAD precursors to generate a synergistic combination in a hydrogel.

In one aspect of the present disclosure, a combination of NAMPT with NMN and NR in hydrogel is shown to regenerate normal skin, with no deficits or scarring, in full thickness skin defects.

In another aspect of this disclosure, a method is described to grow/re-grow hair with a composition having NAMPT and optionally with NAD precursors, such as NMN and NR.

The present disclosure is the first to demonstrate that NAMPT, alone, or in combination with NAD precursors, in which the therapeutic effect is synergistic, is sufficient to regenerate all tissues of the skin. The inventors have shown this in non-diabetic, diabetic and aged-diabetic splinted full thickness excisional skin defect models to recapitulate human skin defect pathophysiology. The aged, diabetic, and aged-diabetic states are characterized by significantly decreased capacity to regenerate skin, and decreased capacity to generate vascularization at the site of skin defect.

In one aspect of this disclosure, a wound healing composition is described. The composition comprises of nicotinamide phosphorylribosyltransferase (NAMPT) protein, an active peptide fragment thereof, or a NAMPT analog, and a pharmaceutically acceptable topical carrier.

In another aspect of this disclosure, a method of treating a skin wound is described. The method comprises administering a therapeutically effective amount of the composition herein described to the skin wound of a subject.

In another aspect of this disclosure, a method for stimulating hair growth and/or regrowth in a subject is described. The method comprises contacting a target region of skin of the subject with an effective amount of a hair growth or regrowth composition, wherein the composition comprises a nicotinamide phosphorylribosyltransferase (NAMPT) protein, an active peptide fragment thereof, a NAMPT analog, a vector comprising a polynucleotide encoding the same, or a combination thereof.

As described in the method herein, the composition comprises a NAMPT protein according to SEQ ID NO: 1.

As described in the method herein, the composition comprises a NAMPT analog that is at least 70% identical to SEQ ID NO: 1.

As described in the method herein, the composition comprises a NAMPT activator, and the NAMPT activator is 3,6-dibromo-α-[(phenylamino)methyl]-9H-carbazol-9-ethanol (P7C3), 1-[4-(8-Oxa-3-azabicyclo[3.2.1]octane-3-sulfonyl)-phenyl]-3-pyridin-4-ylmethylurea (SBI-797812), or combinations thereof.

As described in the method herein, the composition comprises both a NAMPT protein and a NAMPT activator.

As described in the method herein, the composition comprises a vector comprising RNA encoding a NAMPT protein, and the NAMPT protein is according to SEQ ID NO: 1.

As described in the method herein, the composition further comprises a NAD precursor, and the NAD precursor is selected from the group consisting of: tryptophan, nicotinic acid (pyridine-3-carboxylic acid), nicotinamide (nicotinic acid amide), nicotinamide mononucleotide (NMN) and nicotinamide riboside (NR).

As described in the method herein, the composition further comprises a pharmaceutically acceptable carrier, and the pharmaceutically acceptable carrier comprises a topical formulation.

As described in the method herein, the pharmaceutically acceptable carrier comprises a hydrogel. As described in the method herein, the hydrogel comprises a thermosensitive hydrogel. As described in the method herein, the thermosensitive hydrogel is pluronic-F127.

As described in the method herein, the target region of skin is alopecia-affected skin. As described in the method herein, the alopecia-affected skin is part of all of the scalp of the subject.

As described in the method herein, the method further comprises the step of facilitating penetration of the skin of the target region using a skin penetration enhancer selected from needles, abrasive materials, or the application of high pressure to the skin.

As described in the method herein, the method further comprises the step of creating a border around the target region to restrict activity of the NAMPT to substantially within the target region.

As described in the composition herein, the topical carrier is a hydrogel. In one embodiment, the hydrogel may comprise a thermosensitive hydrogel. In one embodiment, the thermosensitive hydrogel is pluronic-F127.

As described in the composition herein, the composition may further comprise a NAMPT activator. In one embodiment, the NAMPT activator is 3,6-dibromo-α-[(phenylamino)methyl]-9H-carbazol-9-ethanol (P7C3), 1-[4-(8-Oxa-3-azabicyclo[3.2.1]octane-3-sulfonyl)-phenyl]-3-pyridin-4-ylmethylurea (SBI-797812), or combinations thereof.

As described in the composition herein, the composition may further comprise a NAD precursor. In one embodiment, the NAD precursor is selected from the group consisting of: tryptophan, nicotinic acid (pyridine-3-carboxylic acid), nicotinamide (nicotinic acid amide), nicotinamide mononucleotide (NMN) and nicotinamide riboside (NR).

As described in the method of treating a skin wound, wherein the skin wound is a diabetic skin wound.

As described in the method of treating a skin wound, wherein the composition comprises at least 0.01 μg (micrograms) of NAMPT protein for every 1 mm² of skin wound surface area to promote complete skin regeneration. In one embodiment, the composition comprises at least 0.05 μg (micrograms) of NAMPT protein for every 1 mm² of skin wound surface area to promote complete skin regeneration.

As used herein, the terms “administer,” “administration,” “administering,” and the like, when used in conjunction with a therapeutic agent means to deliver a therapeutic agent to a subject whereby the therapeutic agent positively impacts, i.e., has a therapeutic effect on, the subject or the tissue or the organ to which it is targeted. The therapeutic agents described herein can be administered either alone or in combination (concurrently or serially) and/or with other pharmaceuticals. For example, the therapeutic agents can be administered in combination with vaccines, antibiotics, antiviral agents, anti-cancer or anti-neoplastic agents, or in combination with other treatment modalities such as herbal therapy, acupuncture, naturopathy, etc.

As used herein, the term “effective amount” generally refers to an amount of the therapeutic agent that is administered to decrease, prevent or inhibit the disease. The amount will vary for each compound and upon known factors related to the item or use to which the therapeutic agent is applied.

As used herein, the term “immune response” refers to activity of the cells of the immune system upon exposure to a stimulus such as but not limited to an antigen. In some embodiments, the antigen may be derived from Bartonella spp.

As used herein, the term “modulation” refers to up regulation (i.e., activation or stimulation), down regulation (i.e., inhibition or suppression) of a response, or the two in combination or apart.

As used herein, the term “pharmaceutically acceptable” refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier”, as used herein, refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

A pharmaceutically acceptable carrier includes any of the many vehicles known and used in the art for delivering drugs, pharmaceuticals and the like to a subject. Herein, a pharmaceutically suitable carrier may be especially appropriate for delivery in topical applications, including without limitation hydrogels, creams, lotions, shampoos, emollients, 85% ethanol/15% ethylene glycol, salves, sprays, oils, dressings, pastes, drops, ointments, liposomes, and the like.

As used herein, the term “prodrug” refers to an agent, including a compound, nucleic acid or protein that is converted into a biologically active form in vitro and/or in vivo. Prodrugs can be useful because, in some situations, they may be easier to administer than the parent compound. For example, a prodrug may be bioavailable by oral administration whereas the parent compound is not. The prodrug may also have improved solubility in pharmaceutical compositions compared to the parent drug. A prodrug may be converted into the parent drug by various mechanisms, including enzymatic processes and metabolic hydrolysis. Harper, N.J. (1962) Drug Latentiation in Jucker, ed. Progress in Drug Research, 4:221-294; Morozowich et al. (1977) Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977) Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997) Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of +-Lactam antibiotics, Pharm. Biotech. 11:345-365; Gaignault et al. (1996) Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogs, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996) Improved oral drug delivery: solubility limitations overcome by the use of prodrugs, Adv. Drug Delivery Rev. 19(2): 115-130; Fleisher et al. (1985) Design of prodrugs for improved gastrointestinal absorption by intestinal enzyme targeting, Methods Enzymol. 112: 360-81; Farquhar D, et al. (1983) Biologically Reversible Phosphate-Protective Groups, J. Pharm. Sci., 72(3): 324-325; Han, H. K. et al. (2000) Targeted prodrug design to optimize drug delivery, AAPS PharmSci., 2(1): E6; Sadzuka Y. (2000) Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl. 2: S15-27; Wang, W. et al. (1999) Prodrug approaches to the improved delivery of peptide drugs. Curr. Pharm. Des., 5(4):265-87.

As used herein, the term “Subject” may include a human subject for medical purposes, such as for the treatment of an existing disease, disorder, condition or the prophylactic for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile and adult subjects. Further a “Subject” can include a patient afflicted with or suspected of being afflicted with a disease, disorder, or condition. Thus, the terms “subject” and “patient” are used interchangeably herein. Subjects also include animal disease models (e.g. rats or mice used in experiments, and the like).

As used herein the term “therapeutic agent” refers to any substance used to restore or promote the health and/or wellbeing of a subject and/or to treat, prevent, alleviate, cure or diagnose a disease, disorder, or condition.

As used herein, the terms “therapeutically effective” and “pharmacologically effective” are intended to quantify the amount of each agent which will achieve the goal of decreasing disease severity while avoiding adverse side effects such as those typically associated with alternative therapies. The therapeutically effective amount may be administered in one or more doses.

The term “effective amount” is intended to quantify the amount of an agent needed to achieve an effect that may not be therapeutic. For example, while hair regrowth may be done primarily for therapeutic purposes, hair growth may be done outside the context of disease, and therefore one can refer to an “effective amount” of agent rather than a “therapeutically effective amount.”

As used herein, the terms “peptide,” “polypeptide” and “protein” are used interchangeably, and refer to a compound comprised of amino acid residues covalently linked by peptide bonds. A protein or peptide must contain at least four amino acids, unless specified otherwise, and no limitation is placed on the maximum number of amino acids that can comprise the sequence of a protein or peptide. Polypeptides include any peptide or protein comprising four or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the terms “treatment,” “treating,” and the like, refer to an intervention performed with the intention of preventing the development or altering the pathology or symptoms of a disorder. Accordingly, “treatment” can refer to therapeutic treatment or prophylactic or preventative measures. In some embodiments, the treatment is for therapeutic treatment. In some embodiments, the treatment is for prophylactic or preventative treatment. Those in need of treatment can include those already with the disorder as well as those in which the disorder is to be prevented. In some embodiments, the treatment is for experimental treatment.

The details of one or more embodiments of the disclosure are set forth in the accompanying description below. Although any materials and methods similar or equivalent to those described herein can be used in the practice or testing of the present disclosure the preferred materials and methods are now described. Other features, objects and advantages of the disclosure will be apparent from the description. In the description the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| EP | European pharmacopoeia |
| NAD | Nicotinamide adenine dinucleotide |
| NAMPT | Nicotinamide phosphoribosyltransferase |
| NR | nicotinamide riboside |
| P7C3 | 3,6-dibromo-α-[(phenylamino)methyl]-9H-carbazol- |
| | 9-ethanol |
| SBI-797812 | 1-[4-(8-Oxa-3- azabicyclo[3.2.1]octane-3-sulfonyl)- |
| | phenyl]-3-pyridin-4-ylmethylurea |
| USP | United States Pharmacopoeia |

### BRIEF DESCRIPTION OF THE DRAWINGS

The foregoing and other features of the present application will become apparent to those skilled in the art to which the present application relates upon reading the following description with reference to the accompanying drawings, in which:

FIGS. 1A-B show (A) intradermal injection of NAMPT every 48 hours in the skin of mice promoted hair growth visibly by 10 and 14 days, as compared to control, while (B) histological analysis revealed increased hair follicles in NAMPT treated mice skin versus control.

FIG. 2A-G. Fibroblast heterogeneity and effect of NAMPT on fibroblasts in diabetic wounds. FIG. 2A: UMAP showing fibroblast populations (Clusters #0, #5, #10, and #12). Gene ontology (GO) was used to determine pathway enrichment of the different fibroblast subpopulations. FIG. 2B: Cluster #0 fibroblasts were enriched for fibroblast proliferation and response to wounding pathways suggesting that Cluster #0 is the major fibroblast cluster of interest in this biological system. FIG. 2C: Pathway enrichment for Cluster #5 fibroblasts. FIG. 2D: Pathway enrichment for Cluster #10 fibroblasts. FIG. 2E: Pathway enrichment for Cluster #12 fibroblasts. FIG. 2F: Violin plots showing differential expression of collagen genes in NAMPT treated diabetic wounds and control diabetic wounds Cluster #0 cells. FIG. 2G: Gene Ontology (GO) analysis reveals Cluster #0 cells from NAMPT treated diabetic wounds are significantly enriched for “Extracellular matrix organization”, and “Response to cytokine”, and “Regulation of cell migration” relative to control diabetic wounds. *** Adjusted p<0.001 by Wilcoxon Sum rank test.

FIG. 3A-C. Effect of NAMPT on undifferentiated epidermal cells in diabetic wounds. FIG. 3A: Violin plots showing differential expression of epidermal stem cell genes in NAMPT treated diabetic wounds and control diabetic wounds Cluster #1 cells. FIG. 3B: Gene Ontology (GO) analysis reveals Cluster #1 cells from NAMPT treated diabetic wounds are significantly enriched for “Response to wounding” and “Regulation of cell migration” pathways relative to control diabetic wounds. FIG. 3C: Pathway analysis confirmed that the functions Cluster #1 undifferentiated epidermal cells included establishment of skin barrier, cell motility, and epithelium development. *** Adjusted p<0.001 by Wilcoxon Sum rank test.

FIG. 4A-D. Pseudotime lineage trajectory analysis reveals NAMPT stimulates epidermal stem cells in diabetic wounds. FIG. 4A: Cells from Cluster #1 were plotted and ordered by pseudotime. FIG. 4B: Trajectory divergence at branchpoint 2 leads to either cell fate 1 or cell fate 2. FIG. 4C. Heatmap demonstrating significantly differentially expressed genes at branchpoint 2 leading to cell fate 1 versus cell fate 2 as a function of pseudotime(q<0.05). Cells from cell fate 1 demonstrate significantly higher levels of Krt5, Krt14, and Krt15. FIG. 4D: Branched analysis shows that the cells from cell fate 1 displaying a rise in Krt5, Krt14, and Krt15 expression belong to NAMPT treated diabetic wounds.

FIG. 5A-D. Pathway analysis reveals differences between NAMPT treated diabetic wounds and control diabetic wounds. FIG. 5A: Plot shows differentially enriched pathways between NAMPT treated diabetic wounds and control diabetic wounds. Pathways enriched between NAMPT treated diabetic wounds are colored red, while pathways enriched in control diabetic wounds are colored green. FIG. 5B: Cellchat clusters are named CLA-CLR, which correspond to clusters #0-#17, respectively. UEC: Undifferentiated Epidermal Cells. DEC: Differentiated Epidermal Cells. VEC: Vascular Endothelial Cells. FIG. 5C: Plot demonstrating WNT signaling between the cell populations of control diabetic wounds. FIG. 5D: Plot shows WNT ligand-receptors in control diabetic wounds.

FIG. 6A-E. NAMPT improves skin regeneration. Excisional full thickness wounds were generated and splinted in nondiabetic mice (A) and diabetic (db/db) mice (B-E) and treated with NAMPT-hydrogel or control vehicle hydrogel. Hematoxylin and Eosin Staining was performed. FIG. 6A-B: Histology revealed regeneration of muscular panniculus carnosus (arrow 1), intradermal adipose (2), dermal appendage morphology (3: sebaceous glands, hair follicles), angiogenesis (4) and lack of dense/scarring-patterns of collagen in NAMPT-hydrogel treated wounds compared to control wounds. FIG. 6C: Immunohistochemistry reveals positive staining for b-catenin and Keratin15 in NAMPT treated diabetic wounds. FIG. 6D: In contrast, control wounds lack b-catenin and Keratin15 staining. FIG. 6E: NAMPT significantly increases percentage of area staining positive for Keratin15 (p=0.0166) and b-catenin (p=0.0332) in diabetic wounds compared to control diabetic wounds (n=3) (t-test).

FIG. 7A-C. Pseudotime lineage trajectory analysis reveals NAMPT stimulates hair follicle formation. FIG. 7A: Cells involved in the Keratin79+ to Krt6+ transition were plotted and ordered by pseudotime. FIG. 7B: Heatmap confirms that the plotted trajectory and pseudotemporal directionality was biologically accurate. FIG. 7C: Keratin79+ progenitor cells and Krt6+ differentiated cells of the hair follicle are present in the NAMPT treated diabetic wounds and absent in control diabetic wounds.

FIG. 8A-C. Pseudotime lineage trajectory analysis reveals NAMPT stimulates sebaceous gland formation. FIG. 8A: Cells involved in sebaceous gland formation were plotted and ordered by pseudotime. FIG. 8B: Heatmap confirms that the plotted trajectory and pseudotemporal directionality was biologically accurate. FIG. 8C: Krt5+/Krt14+ progenitor cells and differentiated Pparg+/Fasn+ cells of sebaceous glands are present in the NAMPT treated diabetic wounds and absent in control diabetic wounds.

FIG. 9A-C. Pseudotime lineage trajectory analysis reveals NAMPT stimulates a Krt77+ cell population. FIG. 9A: Cells involved in the formation of Krt77+ cell population were plotted and ordered by pseudotime. FIG. 9B: Heatmap confirms that the plotted trajectory and pseudotemporal directionality was biologically accurate. FIG. 9C: Krt5+/Krt14+ progenitor cells and differentiated Krt77+ cells are present in the NAMPT treated diabetic wounds and absent in control diabetic wounds.

FIG. 10A-C. Role of NAMPT in epidermal cell differentiation. FIG. 10A: Diagram depicting epidermal differentiation and markers of undifferentiated and differentiated cells. During epidermal cell differentiation, undifferentiated basal cells, characterized by the Krt5+/Krt14+/Krt15+ signature, differentiate into Krt1+/Krt10+ spinous cells, which finally differentiate into terminally differentiated keratinocytes (Cdsn+/Lor+/Ivl+). FIG. 10B: All epidermal cells from non-diabetic skin were plotted and ordered as a function of pseudotime. FIG. 10C: Heatmap confirms that the plotted trajectory and pseudotemporal directionality was biologically accurate by plotting several markers as a function of pseudotime. High Nampt levels were observed in early epidermal stem cells expressing high levels of Krt5, Krt14, and Krt15. The pseudotemporal decrease in Nampt preceded the decreases in stem markers, suggesting Nampt is associated with maintenance of stemness. Interestingly, Nampt expression increased prior to the increase of Krt1 and Krt10, markers of more differentiated epidermal cells, suggesting Nampt is associated with commitment to differentiation. Nampt expression is completely lost in terminally differentiated epidermal cells, which have no intrinsic stemness.

### DETAILED DESCRIPTION

The present invention provides a method for healing wounds in a subject. stimulating hair growth and/or regrowth in a subject. The method includes contacting a target region of skin of the subject with an effective amount of a hair growth or regrowth composition comprising a nicotinamide phosphorylribosyltransferase (NAMPT) protein, an active peptide fragment thereof, a NAMPT analog, a vector comprising a polynucleotide encoding a NAMPT protein, a NAMPT activator, or a combination thereof.

### Formulation

In some embodiments, therapeutic agents are administered to humans, human patients or subjects. For the purposes of the present disclosure, the phrase “active ingredient” generally refers to the therapeutic agents to be delivered as described herein.

Although the descriptions of formulations provided herein are principally directed to formulations which are suitable for administration to humans, it will be understood by the skilled artisan that such therapeutic agents are generally suitable for administration to any other animal, e.g., to non-human animals, e.g., non-human mammals. Modification of formulations suitable for administration to humans in order to render the therapeutic agents suitable for administration to various animals is well understood, and the ordinarily skilled veterinary pharmacologist can design and/or perform such modification with merely ordinary, if any, experimentation. Subjects to which administration of the formulations is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the therapeutic agents described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A formulation in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a formulation in accordance with the disclosure will vary, depending upon the identity, size, and/or condition of the subject treated and further depending upon the route by which the formulation is to be administered. By way of example, the formulation may include between 0.1% and 100%, e.g., between 0.5 and 50%, between 1-30%, between 5-80%, or, in some embodiments, at least 20%, at least 40%, at least 60%, or at least 80% (w/w) active ingredient.

The therapeutic agents of the present disclosure can be formulated using one or more excipients to: (1) increase stability; (2) permit the sustained or delayed release; (3) alter the biodistribution; (4) alter the release profile of the therapeutic agents in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present disclosure may also include, without limitation, lipidoids, liposomes, lipid nanoparticles, polymers, lipoplexes, core-shell nanoparticles, peptides, proteins, hyaluronidase, nanoparticle mimics and combinations thereof.

In some embodiments, pharmaceutical compositions or formulations of the disclosure may be adapted to deliver a prescribed dosage of one or more therapeutic agents to a cell, a group of cells, an organ or tissue, an animal or a human. Methods of incorporating therapeutic agents into pharmaceutical preparations are widely known in the art. The determination of an appropriate prescribed dosage of a pharmacologically active compound to include in a pharmaceutical formulation in order to achieve a desired biological outcome is within the skill level of an ordinary practitioner of the art. The pharmaceutical formulation may include excipients, such as without limitation, binders, coating, disintegrants, fillers, diluents, flavors, colors, lubricants, glidants, preservatives, sorbents, sweeteners, conjugated linoleic acid (CLA), gelatin, beeswax, purified water, glycerol, any type of oil, including, without limitation, fish oil or soybean oil, or the like. Therapeutic agents and/or pharmaceutical formulations can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols. It will further be appreciated by an ordinary practitioner of the art that the term also encompasses those therapeutic agents and/or pharmaceutical formulations that contain an admixture of two or more pharmacologically active compounds, such compounds being administered, for example, as a combination therapy.

A pharmaceutical formulation in accordance with the present disclosure may be prepared, packaged, and/or sold in bulk, as a single unit dose, and/or as a plurality of single unit doses. As used herein, a “unit dose” refers to a discrete amount of the pharmaceutical formulation comprising a predetermined amount of therapeutic agent or other compounds. The amount of therapeutic agent may generally be equal to the dosage of therapeutic agent administered to a subject and/or a convenient fraction of such dosage including, but not limited to, one-half or one-third of such a dosage.

### Excipients

Formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, com starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyllaurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BRA), butylated hydroxytoluened (BHn, ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®11, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphatemonobasic potassium phosphatepotassium phosphate mixturessodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, boragecade, camomile, canola, caraway, camauba, castor, cinnamon, cocoa butter, coconut, cod liver, coffee, com, cotton seed, emu, eucalyptus, evening primrose, fish, flaxseed, geraniol, gourd, grape seed, hazel nut, hyssop, isopropyl myristate, jojoba, kukui nut lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed pumpkin seed, rapeseed, rice bran, rosemary, safflower, sandalwood, sasquana, savoury, sea buckthorn, sesame, shea butter, silicone, soybean, sunflower, tea tree, thistle, Tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### Dosing and Administration

### Administration

In some embodiments, therapeutic agents and/or pharmaceutical formulations that include therapeutic agents may be administered according to one or more administration routes. In some embodiments, administration is enteral (into the intestine), transdermal, intravenous bolus, intralesional (within or introduced directly to a localized lesion), intrapulmonary (within the lungs or its bronchi), diagnostic, intraocular (within the eye), transtympanic (across or through the tympanic cavity), intravesical infusion, sublingual, nasogastric (through the nose and into the stomach), spinal, intracartilaginous (within a cartilage), insufilation (snorting), rectal, intravascular (within a vessel or vessels), buccal (directed toward the cheek), dental (to a tooth or teeth), intratesticular (within the testicle), intratympanic (within the aurus media), percutaneous, intrathoracic (within the thorax), submucosal, cutaneous, epicutaneous (application onto the skin), dental intracornal, intramedullary (within the marrow cavity of a bone), intra-abdominal, epidural (into the dura matter), intramuscular (into a muscle), intralymphatic (within the lymph), iontophoresis (by means of electric current where ions of soluble salts migrate into the tissues of the body), subcutaneous (under the skin), intragastric (within the stomach), nasal administration (through the nose), transvaginal, intravenous drip, endosinusial, intraprostatic (within the prostate gland), soft tissue, intradural (within or beneath the dura), subconjunctival, oral (by way of the mouth), peridural, parenteral, intraduodenal (within the duodenum), intracisternal (within the cisterna magna cerebellomedularis), periodontal, periarticular, biliary perfusion, intracoronary (within the coronary arteries), intrathecal (within the cerebrospinal fluid at any level of the cerebrospinal axis), intrameningeal (within the meninges), intracavemous injection (into a pathologic cavity) intracavitary (into the base of the penis), intrabiliary, subarachnoid, intrabursal, ureteral (to the ureter), intratendinous (within a tendon), auricular (in or by way of the ear), intracardiac (into the heart), enema, intraepidermal (to the epidermis), intraventricular (within a ventricle), intramyocardial (within the myocardium), intratubular (within the tubules of an organ), vaginal, sublabial, intracorporus cavemosum (within the dilatable spaces of the corporus cavemosa of the penis), intradermal (into the skin itself), intravitreal (through the eye), perineural, cardiac perfusion, irrigation (to bathe or flush open wounds or body cavities), in ear drops, endotracheal, intraosseous infusion (into the bone marrow), caudal block, intrauterine, transtracheal (through the wall of the trachea), intra-articular, intracomeal (within the cornea), endocervical, extracorporeal, intraspinal (within the vertebral column), transmucosal (diffusion through a mucous membrane), topical, photopheresis, oropharyngeal (directly to the mouth and pharynx), occlusive dressing technique (topical route administration which is then covered by a dressing which occludes the area), transplacental (through or across the placenta), intrapericardial (within the pericardium), intraarterial (into an artery), interstitial, intracerebral (into the cerebrum), intracerebroventricular (into the cerebral ventricles), intrapleural (within the pleura), infiltration, intrabronchial, intrasinal (within the nasal or periorbital sinuses), intraductal (within a duct of a gland), intracaudal (within the cauda equine), nerve block, retrobulbar (behind the pons or behind the eyeball), intravenous (into a vein), intra-amniotic, conjunctival, intrasynovial (within the synovial cavity of a joint), gastroenteral, intraluminal (within a lumen of a tube), electro-osmosis, intraileal (within the distal portion of the small intestine), intraesophageal (to the esophagus), extra-amniotic administration, hemodialysis, intragingival (within the gingivae), intratumor (within a tumor), eye drops (onto the conjunctiva), laryngeal (directly upon the larynx), urethral (to the urethra), intravaginal administration, intraperitoneal (infusion or injection into the peritoneum), respiratory (within the respiratory tract by inhaling orally or nasally for local or systemic effect), intradiscal (within a disc), ophthalmic (to the external eye), and/or intraovarian (within the ovary).

In some embodiments, therapeutic agents and/or pharmaceutical formulations that include therapeutic agents may be administered by intraarticular administration, extracorporeal administration, intrabronchial administration, endocervical administration, endosinusial administration, endotracheal administration, enteral administration, epidural administration, intra-abdominal administration, intrabiliary administration, intrabursal administration, oropharyngeal administration, interstitial administration, intracardiac administration, intracartilaginous administration, intracaudal administration, intracavemous administration, intracerebral administration, intracorporous cavemosum, intracavitary administration, intracomeal administration, intracisternal administration, cranial administration, intracranial administration, intradermal administration, intralesional administration, intratympanic administration, intragingival administration, intraocular administration, intradiscal administration, intraductal administration, intraduodenal administration, ophthalmic administration, intradural administration, intraepidermal administration, intraesophageal administration, nasogastric administration, nasal administration, laryngeal administration, intraventricular administration, intragastric administration, intrahepatic administration, intraluminal administration, intravitreal administration, intravesicular administration, intralymphatic administration, intramammary administration, intramedullary administration, intrasinal administration, intrameningeal administration, intranodal administration, intraovarian administration, intraperitoneal administration, intrapleural administration, intraprostatic administration, intraluminal administration, intraspinal administration, intrasynovial administration, intratendinous administration, intratesticular administration, subconjunctival administration, intracerebroventricular administration, epicutaneous administration, intravenous administration, retrobulbar administration, periarticular administration, intrathoracic administration, subarachnoid administration, intratubular administration, periodontal administration, transtympanic administration, transtracheal administration, intratumor administration, vaginal administration, urethral administration, intrauterine administration, oral administration, gastroenteral administration parenteral administration, sublingual administration, ureteral administration, percutaneous administration, peridural administration, transmucosal administration, perineural administration, transdermal administration, rectal administration, soft tissue administration, intraarterial administration, subcutaneous administration, topical administration, extra-amniotic administration ear drops, or intravesical infusion.

Therapeutic agents and/or pharmaceutical formulations of the present disclosure may be administered orally but any suitable route of administration may be employed for providing a subject with an effective dosage of drugs of the chemical compositions described herein. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In certain embodiments, it may be advantageous that the compositions described herein be administered orally.

Therapeutic agents and/or pharmaceutical formulations of the present disclosure may be administered in the conventional manner by any route where they are active. Administration can be systemic, parenteral, topical, or oral. For example, administration can be, but is not limited to parenteral, subcutaneous, intravenous, intramuscular, intraperitoneal, transdermal oral, buccal, or ocular routes, or intravaginally, by inhalation, by depot injections, or by implants. Thus modes of administration of the composition of the present disclosure (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

For administration by inhalation or intranasal, pharmaceutical formulation may be delivered in the form of an aerosol spray presentation from pressurized packs or nebulizers. The compounds may also be delivered in the form of a cream, liquid, spray, powder, or suppository. A metered dose of the formulation can be provided from a reservoir of the formulation. In addition, predetermined dosages can be provided, for example, suppository forms can be provided for insertion into the nose having a predetermined dosage. Kits can be provided, where prepared dosage forms and instructions for administering the dosages are included.

Suitable topical formulations for use in the present embodiments may also include transdermal devices, aerosols, creams, ointments, lotions, dusting powders, gels, and the like.

### Dosing

Therapeutic agents and/or pharmaceutical formulations described herein may be administered to a subject using any amount and any route of administration effective treating a disease, disorder, and/or condition. The exact amount required will vary from subject to subject, depending on the species, age, and general condition of the subject, the severity of the disease, the particular formulation, its mode of administration, its mode of activity, and the like

In some embodiments, formulations in accordance with present disclosure may be administered at dosage levels sufficient to deliver a therapeutic agent dose of about 0.1 mg/kg to about 500 mg/kg body weight, from about 0.1 mg/kg to about 250 mg/kg body weight, from about 0.1 mg/kg to about 100 mg/kg body weight, from about 0.1 mg/kg to about 50 mg/kg body weight, from about 0.1 mg/kg to about 10 mg/kg body weight, and/or about 0.1 mg/kg to about 5 mg/kg body weight, from about 1 mg/kg to about 2 mg/kg body weight, from about 1 mg/kg to about 10 mg/kg, from about 5 mg/kg to about 15 mg//kg, from about 10 mg/kg to about 20 mg/kg body weight, from about 20 mg/kg to about 30 mg/kg body weight, from about 30 mg/kg to about 40 mg/kg body weight, from about 40 mg/kg to about 50 mg/kg body weight, from about 50 mg/kg to about 60 mg/kg body weight, from about 60 mg/kg to about 70 mg/kg body weight, from about 70 mg/kg to about 80 mg/kg body weight, from about 80 mg/kg to about 90 mg/kg body weight, from about 90 mg/kg to about 100 mg/kg body weight, from about 100 mg/kg to about 110 mg/kg body weight, from about 110 mg/kg to about 120 mg/kg body weight, from about 120 mg/kg to about 130 mg/kg body weight, from about 130 mg/kg to about 140 mg/kg body weight, from about 140 mg/kg to about 150 mg/kg body weight, from about 150 mg/kg to about 160 mg/kg body weight, from about 160 mg/kg to about 170 mg/kg body weight, from about 170 mg/kg to about 180 mg/kg body weight, from about 180 mg/kg to about 190 mg/kg body weight, from about 190 mg/kg to about 200 mg/kg body weight, from about 15 mg/kg to about 25 mg/kg body weight, from about 25 mg/kg to about 35 mg/kg body weight, from about 35 mg/kg to about 45 mg/kg body weight, from about 45 mg/kg to about 55 mg/kg body weight, from about 55 mg/kg to about 65 mg/kg body weight, from about 65 mg/kg to about 75 mg/kg body weight, from about 75 mg/kg to about 85 mg/kg body weight, from about 85 mg/kg to about 95 mg/kg body weight, from about 95 mg/kg to about 105 mg/kg body weight, from about 105 mg/kg to about 115 mg/kg body weight, from about 115 mg/kg to about 125 mg/kg body weight, from about 125 mg/kg to about 135 mg/kg body weight, from about 135 mg/kg to about 145 mg/kg body weight, from about 145 mg/kg to about 155 mg/kg body weight, from about 155 mg/kg to about 165 mg/kg body weight, from about 165 mg/kg to about 175 mg/kg body weight, from about 175 mg/kg to about 185 mg/kg body weight, from about 185 mg/kg to about 195 mg/kg body weight, from about 195 mg/kg to about 205 mg/kg body weight.

In some embodiments, therapeutic agents described herein may be administered at a dose of about 10-50 μg/mL, 20 μg/mL, or 40 μg/mL.

In some embodiments, therapeutic agents and/or pharmaceutical formulations of the present disclosure are provided in one or more doses and are administered one or more times to subjects. Some therapeutic agents and/or pharmaceutical formulations are provided in only a single administration. Some therapeutic agents and/or pharmaceutical formulations are provided according to a dosing schedule that include two or more administrations. Each administration may be at the same dose or may be different from a previous and/or subsequent dose. In some embodiments, subjects are provided an initial dose that is higher than subsequent doses (referred to herein as a “loading dose”). In some embodiments, doses are decreased over the course of administration. In some embodiments, dosing schedules include pharmaceutical formulation administration from about every 2 hours to about every 10 hours, from about every 4 hours to about every 20 hours, from about every 6 hours to about every 30 hours, from about every 8 hours to about every 40 hours, from about every 10 hours to about every 50 hours, from about every 12 hours to about every 60 hours, from about every 14 hours to about every 70 hours or longer, depending on the need of the subject.

The desired dosage may be delivered for a duration of about 5 days to 365 days, about 5 days to 300 days, about 5 days to 300 days, about 5 days to 250 days, about 5 days to 200 days, about 5 days to 100 days, about 5 days to 60 days, about days to 30 days, about 5 days to 14 days, or about 3 days to 7 days, preferably about 21 days to 28 days.

In some embodiments, the desired dosage of the formulations described herein may be administered once daily or multiple times in a day. For example, a treatment regimen may include administering a dosage level sufficient to deliver 10 mg/kg body weight twice daily, 20 mg/kg body weight twice daily, 50 mg/kg body weight once daily, 10 mg/kg body weight three times daily, 20 mg/kg body weight four times daily, or 50 mg/kg body weight twice daily.

### Combinations

In some embodiments, the therapeutic agents and/or pharmaceutical formulations of the present disclosure may be used in combination with additional active agents such as antibiotics and/or vaccines. By “in combination with,” it is not intended to imply that the agents must be administered at the same time and/or formulated for delivery together, although these methods of delivery are within the scope of the present disclosure.

In some embodiments, the present disclosure encompasses the delivery of pharmaceutical, prophylactic, research, or diagnostic formulations in combination with agents that may improve their bioavailability, reduce and/or modify their metabolism, inhibit their excretion, and/or modify their distribution within the body.

The formulations of the present disclosure and the additional active agents may be administered simultaneously, sequentially or at any order. The formulations of the present disclosure and additional active agents may be administered at different dosages, with different dosing frequencies and/or different routes, whichever is suitable. The term “administered simultaneously”, as used herein, may mean that formulations of the present disclosure and the additional active agent may be substantially administered at the same time, e.g., as a mixture or in immediate subsequent sequence. The term “administered sequentially”, as used herein, may mean that the formulations of the present disclosure and the additional active agent may not be administered at the same time but one after the other, or in groups, with a specific time interval between administrations. The time interval may be the same or different between the respective administrations of the formulations of the present disclosure and the additional active agent and may be selected, for example, from the range of 2 minutes to 96 hours, 1 to 7 days or one, two or three weeks. Generally, the time interval between the administrations may be in the range of a few minutes to hours, such as in the range of 2 minutes to 72 hours, 30 minutes to 24 hours, or 1 to 12 hours.

All scientific and technical terms used in the present application have meanings commonly used in the art unless otherwise specified. The definitions provided herein are to facilitate understanding of certain terms used frequently herein and are not meant to limit the scope of the present application.

### Stimulating Hair Growth or Regrowth

The present invention provides a method for stimulating hair growth and/or regrowth in a subject. The method comprising contacting a target region of skin of the subject with an effective amount of a hair growth or regrowth composition comprising a nicotinamide phosphorylribosyltransferase (NAMPT) protein, an active peptide fragment thereof, a NAMPT analog, a vector comprising polynucleotide encoding a NAMPT protein, a NAMPT activator, or a combination thereof. In some embodiments, the hair growth or regrowth composition comprises a pharmaceutically acceptable carrier.

Contacting, as used herein, refers to causing two items to become physically adjacent and in contact, or placing them in an environment where such contact will occur within a reasonably short timeframe. For example, contacting a site with a composition comprising NAMPT includes administering the composition (e.g., topical administration) to a subject at or near a site such that the NAMPT will interact with the site to stimulate hair growth or regrowth. However, contacting also includes systemic administration which results in contact between NAMPT and the target region through circulation-mediated contact.

Hair growth, as used herein, refers to stimulating the growth of hair in a target region of skin where hair may not have been present before, or where a higher amount of hair is desired simply as a matter of personal choice. Hair regrowth, on the other hand, refers to the restoration of hair levels after hair loss, such as the hair loss caused by alopecia. The amount of hair growth or regrowth stimulated can vary from an increase of the amount of hair of about 10%, about 20%, about 30%, about 40%, about 50%, about 60%, about 70%, about 80%, about 90%, or 100% or more. Hair growth also includes stimulating the growth and/or regeneration of hair follicles.

### Nicotinamide Phosphoribosyltransferase

In some embodiments, the hair growth and regrowth composition comprises the NAMPT protein, an active peptide fragment thereof, or a NAMPT analog. Nicotinamide phosphoribosyltransferase (NAMPT) is s an enzyme that in humans is encoded by the NAMPT gene. Samal et al., Molecular and Cellular Biology. 14 (2): 1431-1437 (1994). NAMPT was previously known as pre-B-cell colony-enhancing factor 1 (PBEF1) or visfatin for its extracellular form (eNAMPT), and is also known as NMN pyrophosphorylase or NMN synthetase. NAMPT is found in all tissues of mammals, and its coding sequence is well conserved. McGlothlin et al., Biochem. Genet. 43, 127-141 (2005). NAMPT has a molecular weight of around 55 kDa and primarily consists of 491 amino acids. Its x-ray crystal structure has been recorded and it recognized as a dimeric class of type II phosphoribosyltransferases.

The amino acid sequence of human NAMPT is provided by SEQ ID NO: 1, shown below. See Samal et al., Mol Cell Biol., 14(2): 1431-1437 (1994).

ORIGIN

| | |
|---|---|
| 1 | mnpaaeaefn illatdsykv thykqyppnt skvysyfecr |
| | ekktensklr kvkyeetvfy |
| 61 | glqyilnkyl kgkvvtkeki qeakdvykeh fqddvfnekg |
| | wnyilekydg hlpieikavp |
| 121 | egfviprgnv lftventdpe cywltnwiet ilvqswypit |
| | vatnsreqkk ilakyllets |
| 181 | gnldgleykl hdfgyrgvss qetagigasa hlvnfkgtdt |
| | vaglalikky ygtkdpvpgy |
| 241 | svpaaehsti tawgkdhekd afehivtqfs svpvsvvsds |
| | ydiynaceki wgedlrhliv |
| 301 | srstqaplii rpdsgnpldt vlkvleilgk kfpvtenskg |
| | ykllppylrv iqgdgvdint |
| 361 | lqeivegmkq kmwsieniaf gsgggllqkl trdllncsfk |
| | csyvvtnglg invfkdpvad |
| 421 | pnkrskkgrl slhrtpagnf vtleegkgdl eeygqdllht |
| | vfkngkvtks ysfdeirkna |
| 481 | qlnieleaah h |
| // | |

NAMPT proteins, peptide fragments thereof, mutants, truncations, derivatives, analogs, and splice variants that display substantially equivalent or altered NAMPT activity relative to the wild-type protein are likewise contemplated for use in the present invention. These variants may be deliberate, for example, such as modifications obtained through site-directed mutagenesis, or may be accidental, such as those obtained through mutations in hosts that are producers of the NAMPT protein. Included within the scope of these terms are NAMPT proteins specifically recited herein, as well as all substantially homologous analogs and allelic variants.

Analogs may be made through substitution of conserved amino acids. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in an NAMPT protein is preferably replaced with another amino acid residue from the same side chain family. Alternatively, in another embodiment, mutations can be introduced randomly along all or part of an NAMPT coding sequence, such as by saturation mutagenesis, and the resultant mutants can be screened for activity to identify mutants that retain activity. Following mutagenesis of the nucleotide sequence for NAMPT, the encoded protein can be expressed recombinantly and the activity of the protein can be determined. In some embodiments, the composition comprises a NAMPT analog that is at least 70% identical to SEQ ID NO: 1.

A “non-essential” amino acid residue is a residue that can be altered from the wild-type sequence of GPNMB without abolishing or, more preferably, without substantially altering a biological activity, whereas an “essential” amino acid residue results in such a change. For example, amino acid residues that are conserved among the polypeptides of the present invention are predicted to be particularly unamenable to alteration.

As used herein, an “active peptide fragment” of a NAMPT protein includes a fragment of a NAMPT protein that retains enzymatic activity. Biologically active portions of a NAMPT protein include peptides comprising amino acid sequences sufficiently homologous to or derived from the amino acid sequence of a NAMPT protein which include less amino acids than a full length GPNMB proteins and which exhibit at least one activity of an GPNMB protein. A biologically active portion of a GPNMB protein can be a polypeptide which is, e.g., 50, 100, 200, or 300 or more amino acids in length.

In some embodiments, the composition comprises a NAMPT activator. NAMPT activators are compounds that increase the activity of NAMPT. A number of NAMPT activators have been identified. See Wang et al., Eur J Med Chem., 236:114260 (2022). In some embodiments, the NAMPT activator is selected from 3,6-dibromo-α-[(phenylamino)methyl]-9H-carbazol-9-ethanol (P7C3) (Wang et al., Cell, 158(6), 1324-1334 (2014)) and 1-[4-(8-Oxa-3-azabicyclo[3.2.1]octane-3-sulfonyl)-phenyl]-3-pyridin-4-ylmethylurea (SBI-797812). In some embodiments, the composition comprises a NAMPT protein and a NAMPT activator.

In some embodiments, the composition wherein the composition further comprises a nicotinamide adenine dinucleotide (NAD) precursor. NAD precursors include tryptophan, nicotinic acid (pyridine-3-carboxylic acid), nicotinamide (nicotinic acid amide), nicotinamide mononucleotide (NMN) and nicotinamide riboside (NR).

Candidate agents may be tested in animal models. Typically, the animal model is one for the study of hair growth. The study of hair growth in animal models (for instance, mice) is a commonly accepted practice for the study of human hair growth or regrowth. Results are typically compared between control animals treated with candidate agents and the control littermates that did not receive treatment. Candidate agents can be used in these animal models to determine if a candidate agent increases the rate of hair growth or regrowth. Candidate agents can also be evaluated for their ability to increase NAMPT activity.

In some embodiments, the composition comprises a vector comprising a polynucleotide encoding a NAMPT protein. Examples of vectors include viral vectors and plasmids. The polynucleotide can be a DNA sequence or an RNA sequence. In further embodiments, the NAMPT protein encoded by the polynucleotide is according to SEQ ID NO: 1.

A wide variety of pharmaceutically acceptable carriers are described herein. In some embodiments, the pharmaceutically acceptable carrier comprises a hydrogel. The hydrogel can include a hydrogel formed from a variety of different polymers. In the medical field, many proprietary hydrogel formulations are known. In certain embodiments, hydrogel sheets of cross-linked polymer gels are used. It is contemplated that hydrogels from various sources will find use with the present methods and compositions, including, but not limited to commercially available hydrogels.

In some embodiments, the hydrogel comprises a thermosensitive hydrogel.

Thermosensitive hydrogels are aqueous polymer solutions that are transformed into gels by changes in environmental temperature, thus resulting in in situ hydrogel formation. Examples of thermosensitive hydrogels include chitosan and related derivatives, poly(N-isopropylacrylamide)-based (PNIPAAM) copolymers, poly(ethylene oxide)/poly(propylene oxide) (PEO/PPO) copolymers and its derivatives, and poly(ethylene glycol)/biodegradable polyester copolymers. Gong et al., Curr Med Chem20(1):79-94 (2013). In some embodiments, the thermosensitive hydrogel is pluronic-F127, also known as poloxamer 407, which is a triblock copolymer consisting of a central hydrophobic block of polypropylene glycol flanked by two hydrophilic blocks of polyethylene glycol (PEG).

The method of stimulating hair growth or regrowth includes the step of contacting a target region of skin of the subject with an effective amount of the hair regrowth composition. In some embodiments, the target region of skin is alopecia-affected skin. Hair, as is well known, are specialized keratinized structures derived or protruding from invaginations of the epidermal epithelium that are observed on animals, including mammals, and includes fur.

Hair loss (i.e., baldness or hair thinning) from areas where hair is normally present. Alopecia encompasses hair loss that results from any cause. Hair loss also includes as hair thinning, baldness, male and female pattern baldness, thinned eye lashes, and thinned eye brows. The term encompasses full or partial hair loss, shedding or any decrease in the number of follicles or follicles in the anagen phase at any body site where hair is normally present. In some embodiments, the alopecia-affected skin is part or all of the scalp of the subject.

In some embodiments, the method further comprises the step of facilitating penetration of the skin of the target region using a skin penetration enhancer. Skin penetration enhancers include both physical and chemical skin penetration enhancers. For example, physical skin penetration enhancers can be selected from needles (e.g., a manifold of needles), abrasive materials, or the application of high pressure to the skin. Alternately, a chemical skin penetration enhancer can be used. Examples of chemical skin penetration enhancers include glyceryl oleate (glycerol monooleate); isopropyl myristate; methyl laurate; N-lauroyl sarcosine; oleic acid (octadecenoic acid); sodium lauryl sulfoacetate; and sodium octyl sulfate. The skin penetration enhancer can be applied to the target region to facilitate penetration of NAMPT, as well as an active peptide fragment thereof, a NAMPT analog, a vector comprising RNA encoding a NAMPT protein, or a NAMPT activator, through the skin at the target region.

In some embodiments, the method further comprises the step of creating a border around the target region to restrict activity of the NAMPT to substantially within the target region. The border corresponds to the periphery of the target region, or the dividing line between the target region and non-target regions of the skin. The border can be created using physical or chemical means to prevent NAMPT, as well as an active peptide fragment thereof, a NAMPT analog, a vector comprising RNA encoding a NAMPT protein, or a NAMPT activator, from passing beyond the target region, thereby preventing an increase of NAMPT activity beyond the target region. For example, the border can be formed by placement of compounds that inhibit NAMPT such as small molecule inhibitors or antibodies against NAMPT in the border.

### Administration of NAMPT

The present invention includes contacting a target region with an effective amount of a hair growth or regrowth composition comprising NAMPT protein, an active peptide fragment thereof, a NAMPT analog, a vector comprising polynucleotide encoding a NAMPT protein, a NAMPT activator, or a combination thereof. The target region can be contacted with composition as a result of systemic or local administration to the subject.

As used herein, the terms “localized” and “local” refer to the involvement of a limited area. Thus, in contrast to “systemic” treatment, in which the entire body is involved, usually through the vascular and/or lymph systems, localized treatment involves the treatment of a specific, limited area. Thus, in some embodiments, regions are treated locally using the methods and compositions of the present invention. In some embodiments, the pharmaceutically acceptable carrier comprises a topical formulation. Topical administration refers to application to the surface of the skin, mucosa, viscera, etc.

In some embodiments, the hair regrowth composition includes a pharmaceutically acceptable carrier to facilitate administration. The active agent (e.g., NAMPT or NAMPT agents) is preferably utilized together with one or more pharmaceutically acceptable carrier(s) and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect (i.e., hair growth or regrowth), and in a quantity appropriate to achieve the desired daily dose.

Typically, the NAMPT will be suspended in a sterile saline solution for therapeutic uses. The pharmaceutical compositions may alternatively be formulated to provide sustained release of NAMPT locally or systemically. Numerous suitable drug delivery systems are known and include, e.g., implantable drug release systems, hydrogels, hydroxymethylcellulose, microcapsules, liposomes, microemulsions, microspheres, and the like.

The pharmaceutical composition of this invention may be administered by any suitable means, such as orally, topically, intradermally, subcutaneously, intranasally, subcutaneously, intramuscularly, intravenously, intra-arterially, or parenterally. Ordinarily, topical administration may be preferred. When administered, the hair growth or regrowth compositions may be combined with other ingredients, such as carriers and/or adjuvants. The NAMPT or other active ingredient(s) may also be covalently attached to a protein carrier, such as albumin, so as to decrease metabolic clearance of the peptides.

It will be apparent to those of ordinary skill in the art that the therapeutically effective amount of NAMPT will depend, inter alia upon the administration schedule, the unit dose of molecule administered, whether the peptide is administered in combination with other therapeutic agents, the immune status and health of the patient, the therapeutic activity of the peptide administered and the judgment of the treating physician.

Although an appropriate dosage of a molecule of the invention varies depending on the administration route, age, body weight, sex, or conditions of the subject, and should be determined by the physician in the end, in the case of oral administration, the daily dosage can generally be between about 0.01 mg to about 500 mg, preferably about 0.01 mg to about 50 mg, more preferably about 0.1 mg to about 10 mg, per kg body weight. In the case of parenteral administration, the daily dosage can generally be between about 0.001 mg to about 100 mg, preferably about 0.001 mg to about 10 mg, more preferably about 0.01 mg to about 1 mg, per kg body weight. The daily dosage can be administered, for example in regimens typical of 1-4 individual administration daily. Dosage administered can also be measured by using a target serum concentration. For example, in some embodiments, a dosage can be administered to provide a serum concentration from 100 ng/mL. to 1000 ng/mL, from 200 ng/mL to 800 ng/mL, from 300 ng/mL to 500 ng/mL, or at least 400 ng/mL. Various considerations in arriving at an effective amount are described, e.g., in Goodman and Gilman's: The Pharmacological Bases of Therapeutics, 8th ed., Pergamon Press, 1990; and Remington's Pharmaceutical Sciences, 17th ed., Mack Publishing Co., Easton, Pa., 1990.

The NAMPT can be dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those skilled in the art. In addition, if desired, the composition can contain minor amounts of auxiliary substances such as wetting or emulsifying agents, pH buffering agents.

In some embodiments, a single dose of NAMPT is administered. However, in other embodiments, the NAMPT, NAMPT analog, NAMPT active peptide fragment, vector comprising RNA encoding a NAMPT protein, or NAMPT activating agent is administered repeatedly or continuously over a significant period of time. This can be achieved either through repeated administration, or through use of a sustained-release formulation.

The following example is for the purpose of illustration only and is not intended to limit the scope of the claims, which are appended hereto.

### Example 1: Nampt Promotion of Hair Growth

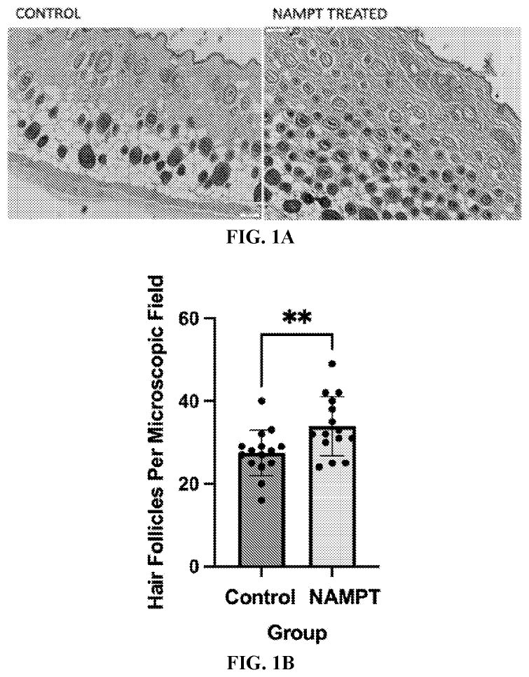

Intradermal injection of NAMPT every 48 hours in the skin of mice promoted hair growth visibly by 10 and 14 days, as compared to control (FIG. 1A). Histological analysis revealed increased hair follicles in NAMVPT treated mice skin versus control (FIG. 1B).

For quantification of FIG. 1A, as shown in FIG. 1B, hair follicles were counted from 15 fields at 200× magnification from each group manually (by eye) in unblinded manner. There was a significant increase in number of hair follicles in the NAMPT group versus control. The mean number of hair follicles per field in the NAMiPT group is 33.93. The mean number of hair follicles per field in the control group is 27.47. The difference between the number of hair follicles per field between the NAMPT group and control group is statistically significant (p-value 0.0097 by unpaired t-test (GraphPad Prism 9).

Unpaierd t test

| | |
|---|---|
| P value | 0.0097 |
| P value summary | ** |
| Significantly different (P < 0.05)? | Yes |
| One- or two-tailed P value? | Two-tailed |
| t, df | t = 2.777, df = 28 |

How big is the difference?

| | |
|---|---|
| Mean of column A | 27.47 |
| Mean of column B | 33.93 |
| Difference between means (B − A) ± SEM | 6.467 ± 2.329 |
| 95% confidence interval | 1.697 to 11.24 |
| R squared (eta squared) | 0.2160 |

F test to compare variances

| | |
|---|---|
| F, DFn, Dfd | 1.662, 14, 14 |
| P value | 0.3530 |
| P value summary | ns |
| Significantly different (P < 0.05)? | No |

Data analyzed

| | |
|---|---|
| Sample size, column A | 15 |
| Sample size, column B | 15 |

The complete disclosure of all patents, patent applications, and publications, and electronically available materials cited herein are incorporated by reference. Any disagreement between material incorporated by reference and the specification is resolved in favor of the specification. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

### Nampt and Wound Healing

Diabetes mellitus is a systemic disease that affects multiple organs, including the skin. Approximately one-third of patients with diabetes have skin disorders. Noninfectious skin disorders in diabetic patients include pruritis, necrobiosis lipoidica, granuloma annulare, and scleredema of Bucshke (scleroderma diabetorum). Diabetic skin is also more susceptible to bacterial and fungal infections. Delayed wound healing and compromised skin regeneration are notable complications of diabetes mellitus. The underlying pathophysiology, including vasculopathy (microangiopathy), neuropathy, biochemical alterations, and altered immunity, results in altered tissue homeostasis and impairs repair and regeneration of diabetic skin during wound healing.

Undifferentiated epidermal cells and fibroblasts in diabetic skin have deficiencies compared to nondiabetic skin, which may underly impaired wound healing. Decreased stem cell markers in undifferentiated epidermal cells suggest an impairment in regenerative potential of resident epidermal cells. Decrease in collagen expression in fibroblasts suggest that diabetic fibroblasts may not be primed for rapid granulation tissue formation during wound healing. Our analysis also showed that diabetic skin is deficient in several metabolic pathways relative to nondiabetic skin, including oxidative phosphorylation, glutathione metabolism, and glycolysis.

### Experimental Methods

Diabetic, aged diabetic, and nondiabetic mice were purchased from JAX labs. Bilateral, symmetric full thickness wounds (which recapitulate complete skin tissue loss) were generated on the murine dorsum using a biopsy punch; wounds were treated with the disclosed therapeutic or the control vehicle.

The inventors successfully synthesized and optimized hydrogels containing combinations of recombinant NAMPT, NR and NMN. A broad range of concentrations for NAMPT, NR and NMN were prepared in hydrogel.

We utilize a splinted, full thickness excisional wound model in diabetic db/db mice and wildtype mice and demonstrated that NAMPT promotes skin regeneration, with restoration of normal skin architecture and components. NAMPT hydrogel was administered on wounds, control hydrogel served as control.

In both diabetic and nondiabetic wounds, increased vascular density, vascular loops and nets were grossly visible in the treated wounds, compared to controls. Histology revealed near normal skin architecture in the treated wounds, with proper epithelial thickness, non-scarring, basket-weave patterns of collagen, reappearance of dermal adipose, regeneration of the muscular panniculus carnosus, and even early regeneration of sebaceous glands, that resembled adjacent non-wound tissue. In contrast, the control wounds showed disorganized, scarring pattern of collagen, lack of adipose and sebaceous glands, and abnormal thicker epidermal hyperplasia.

In both diabetic and nondiabetic wounds, tissue analysis indicated that NAMPT-hydrogel treatment promotes skin regeneration including regeneration of hair follicles, sebaceous glands, adipose tissue, the muscular panniculus carnosus and non-scarring patterns of collagen. NAMPT-hydrogel also promotes robust angiogenesis in both non-diabetic and diabetic mouse wounds, promoting regeneration of the newly forming skin. Immunohistochemistry revealed significantly increased Keratin15 and B-catenin in NAMPT treated diabetic wounds, compared with control diabetic wounds, which is consistent with our scRNAseq findings. The increased B-catenin suggests increased Wnt signaling activation. These findings confirm our scRNAseq findings, including increased epidermal stem cell populations and activity, as well as lineage analyses suggesting enhanced sebaceous gland and hair follicle formation. We observed basket weave collagen deposition and lack of scarring in the NAMPT treated wounds, which is confirmed by increased activity of wound responsive fibroblasts and regenerative signaling such as Wnt, as demonstrated by scRNAseq.

As shown above, in both diabetic and nondiabetic wounds, NAMPT hydrogel promoted complete skin regeneration; the regenerated tissue was indistinguishable from adjacent skin, including full hair follicle and gland regeneration. Gross examination revealed significantly more and larger vascularization in the treated wound, present in the dermis, and also extending from the subcutaneous tissue to the wound bed, suggesting the disclosed treatment also promoted subcutaneous tissue involvement in skin regeneration. Further, the treated wounds contained a higher amount of subcutaneous fat, relative to control. Control wounds showed marked inflammation and thick dense scarring collagen pattern, and minimal dermal adipose formation, minimal follicles, and minimal glands.

In diabetic mice, treatment with NAMPT hydrogel results in increased vascularity and granulation tissue. complete regeneration of skin architecture is present, including regenerated dermis (non-scarring dermis) with glands, vasculature, and hair follicles and intradermal adipose tissue. Control diabetic wounds have little to no dermis formation and minimal tissue regeneration. The inventors are the first to show that local NAMPT can rescue impaired diabetic skin regeneration, including angiogenesis and granulation formation and re-epithelialization.

The inventors are the first to demonstrate that NAMPT, alone, or in combination with NAD precursors, is sufficient to regenerate all tissues of the skin in both non-diabetic and diabetic states. The inventors are the first to formulate combinations of NAMPT, NR, and NMN within clinically deployable hydrogels for complete skin regeneration without scarring.

Novel NAMPT containing hydrogels promote increased vascular density and loops, result in complete skin regeneration with glands, hair follicles, blood vessels, adipose, muscle, basket-weave collagen, and inhibit scarring-pattern collagen formation.

The novel NAMPT-containing hydrogel resulted in complete skin regeneration with glands, vasculature, and hair follicles and intradermal adipose tissue.

### Example 3: High Efficiency Delivery of NAMPT

The inventors are further developing clinically deployable methods to deliver the disclosed treatment, including testing novel high efficiency in vivo lipid nanoparticles carrying codon-optimized NAMPT mRNA, alone, and in combination with PDGF, VEGF, EGF, SIRT1, and other mRNA. These lipid nanoparticles are injected at the site of skin defect or are embedded in hydrogels for sustained release.

### Example 4: Nampt Formulation with NR and NMN

The inventors are also testing NR and NMN liposomal formulations within hydrogels, and in combination with mRNA delivery.

The results will likely show that combination treatments will be viable and equally or even perhaps more effective, working through a similar mechanism of action.

### Example 5: NAMPT Formulation with Activators

The inventors are also developing the first in class NAMPT+P7C3+SBI797812 (NAMPT activator) combination and P7C3 and SBI-797812 mono- and duo-formulations that will prime endogenous and exogenous, and intracellular and extracellular NAMPT to maximally increase NAD. These combinations with NR and NMN are also being developed.

The results will likely show that combination treatments will be viable and equally or even perhaps more effective, working through a similar mechanism of action.

### Example 6: Regulating In Vivo Expression of NAMPT

Finally, the inventors are optimizing lentiviral based NAMPT delivery technology for regulatable expression locally and systemically. The inventors anticipate these technologies will provide significantly accelerated regeneration of skin, and all tissues within, with no deficit and no scarring.

We produce lentiviral vector based therapies based on standard protocols. The lentiviral based NAMPT delivery technology for regulatable expression locally and systemically. The technologies are likely to provide significantly accelerated regeneration of skin and tissues within with no deficit and no scarring.

Studies including single-cell RNA sequencing performed by the authors have elucidated the mechanism of action of the disclosed novel treatment on complete skin regeneration and prevention of scarring.

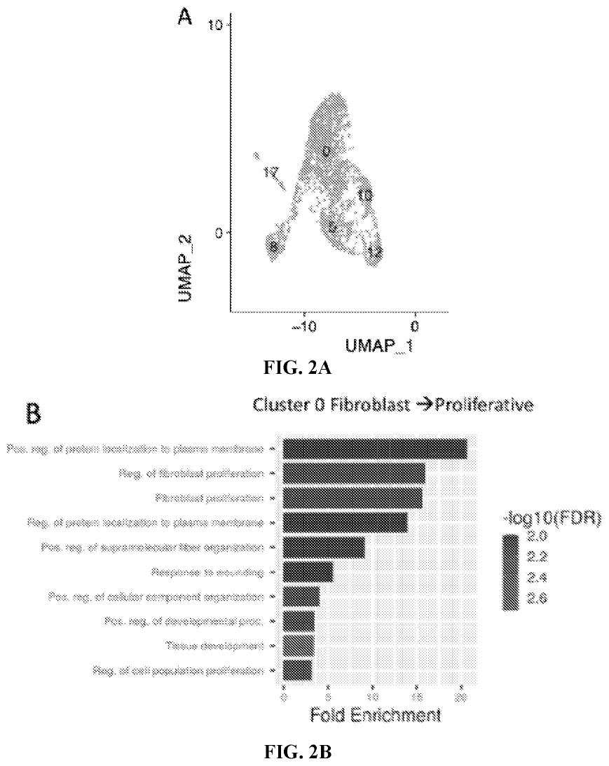

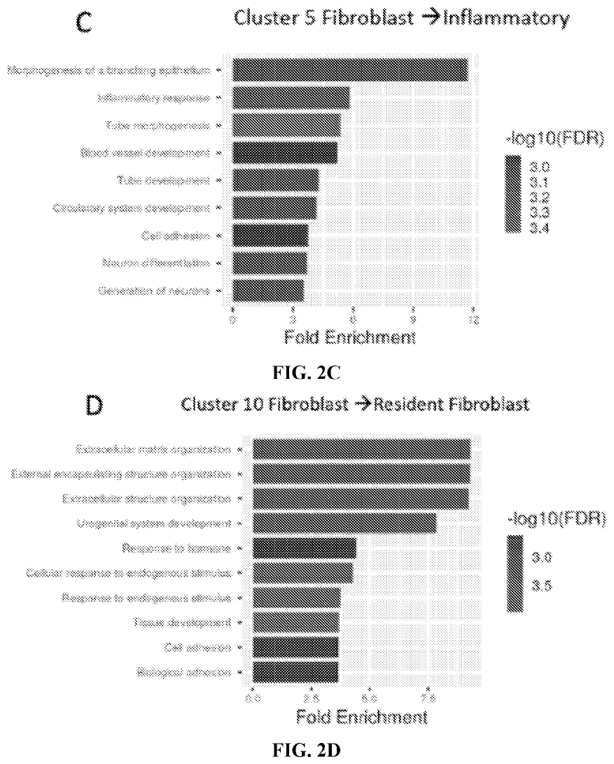

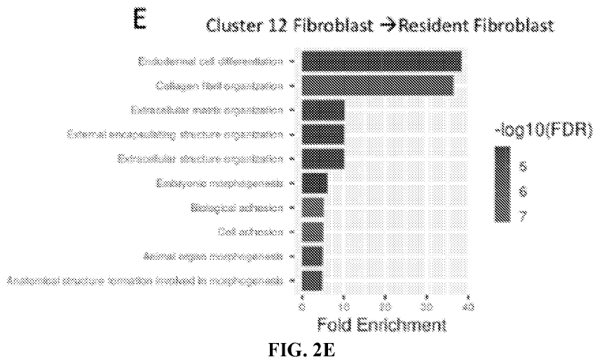

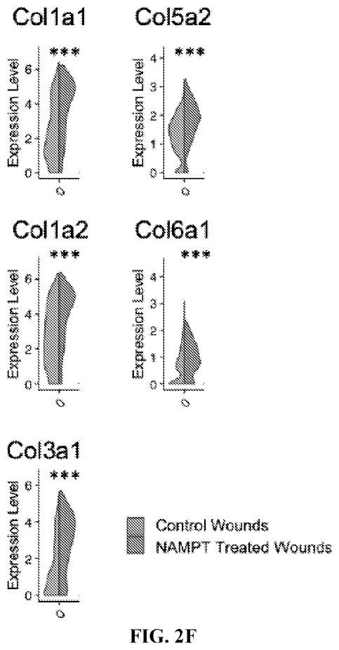

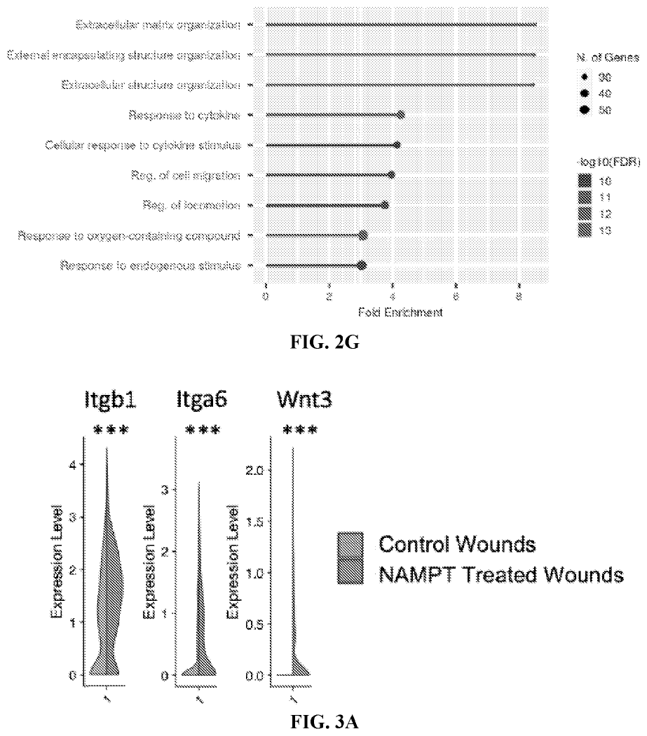

We performed gene ontology (GO) analysis on differentially expressed genes between the fibroblast clusters (clusters #0, #5, #10, and #12) to determine whether specific functions could be assigned to the different fibroblast subpopulations (FIG. 2A-E). Cluster #0 was enriched for fibroblast proliferation and response to wounding pathways, suggesting that Cluster #0 is the major fibroblast cluster of interest in this biological system (FIG. 2B). Next, we performed gene ontology analysis on differentially expressed genes between NAMPT treated versus control Cluster #0 fibroblasts. NAMPT treated Cluster #0 fibroblasts were enriched for extracellular matrix organization, response to cytokine, and regulation of cell migration and motility pathways, suggesting that treatment with NAMPT mobilizes this population of fibroblasts in diabetic wounds (FIG. 2G). Consistently, gene expression for collagens including Col1a1, Col1a2, Col3a1, Col5a2, and Col6a1 were all significantly increased in NAMPT treated Cluster #0 fibroblasts (FIG. 2F).

### Effect of NAMPT on Undifferentiated Epidermal Cells in Diabetic Wounds

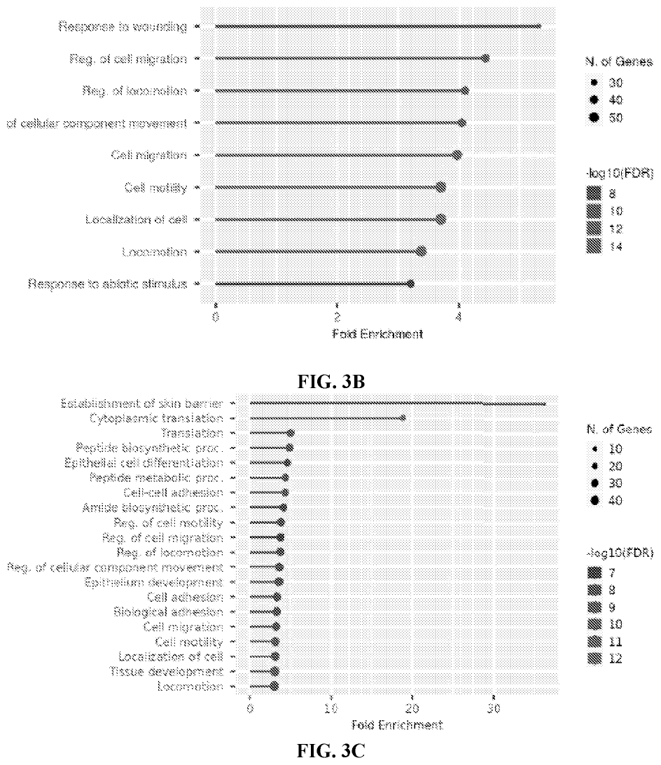

Beyond marker-based classification, we confirmed that Cluster #1 represents undifferentiated epidermal cells through pathway enrichment. Pathway analysis showed that the gene expression signature of this cluster was involved in establishment of skin barrier, cell motility, and epithelium development (FIG. 3C). We performed gene ontology (GO) analysis on Cluster #1 undifferentiated epidermal cells, which is the largest cluster of undifferentiated epidermal cells. This revealed that NAMPT treated wounds were enriched for response to wounding and cell migration, suggesting that NAMPT treatment mobilizes undifferentiated epidermal cells in the wound environment (FIG. 3B). Further, NAMPT treated Cluster #1 cells expressed significantly higher epidermal stem cell markers, including Krt5, Krt14, Krt15, Trp63, Itgb1, and Itga6, compared to controls (FIG. 3A). Trp63 is a p53 homolog that identifies epidermal stem cells12. Itga6 is also a marker of epidermal stem cells. Altogether, these suggest that NAMPT is enhancing epidermal stem cell populations and their reparative function in diabetic wounds.

### Pseudotime Lineage Trajectory Analysis Reveals NAMP T Stimulates Epidermal Stem Cells in Diabetic Wounds

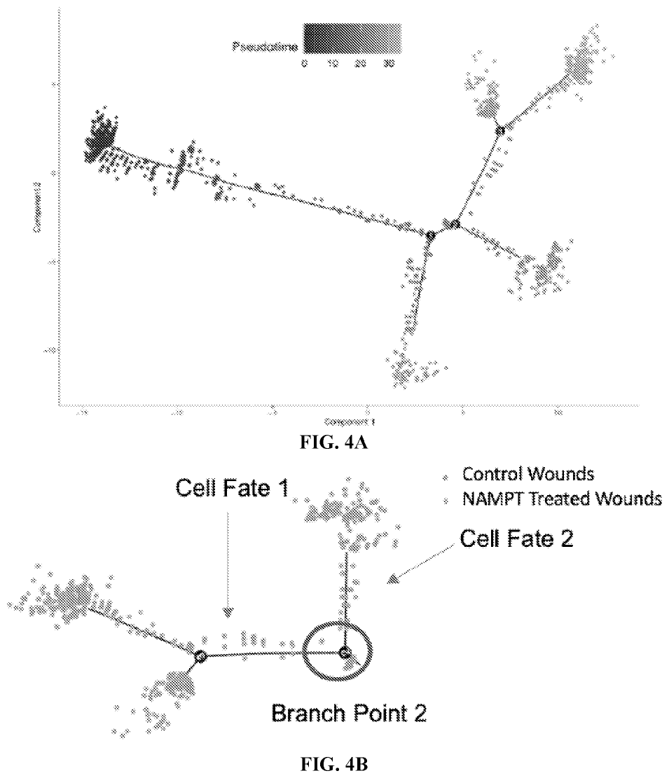

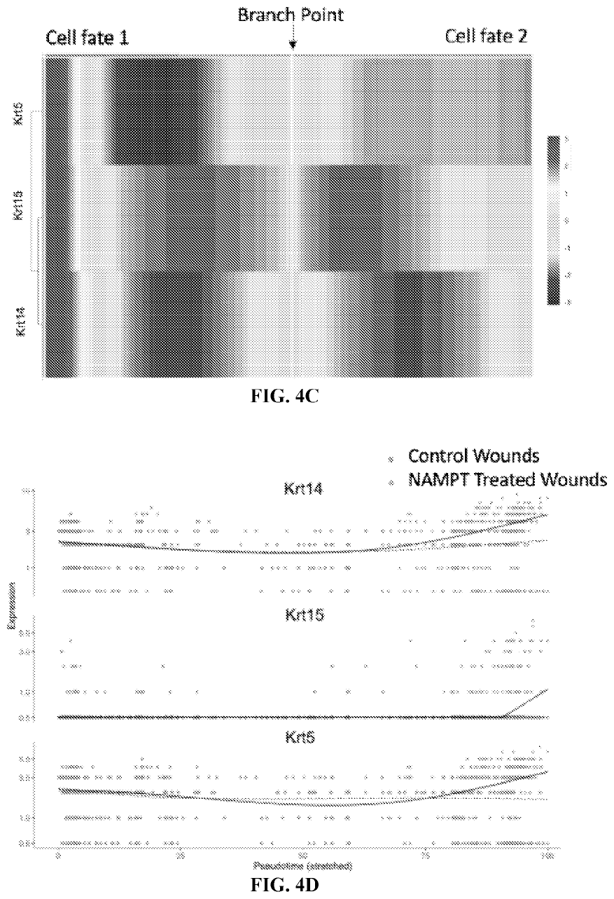

To further confirm that NAMPT treatment stimulated epidermal stem cells in diabetic model, we performed lineage trajectory analysis of Cluster #1 cells. After plotting cells and ordering by pseudotime, we identified branch point 2 as a terminal branchpoint leading to two cell fates (FIG. 4A-D). Cells from NAMPT treated wounds dominate cell fate 1, while cells from control wounds dominate cell fate 2 (FIG. 4B). The heatmap demonstrates that cells from NAMPT treated wounds expressed significantly higher levels of Krt5, Krt14, and Krt15, suggesting an enriched epidermal stem cell population (FIG. 4C). The genes plotted as a function of pseudotime along the two cell fates revealed and confirmed that the majority of cells with increased Krt5, Krt14, and especially Krt15 expression were induced only in NAMPT treated wounds, and not the control wounds (FIGS. 4C and 4D). The pseudotemporal directionality of the stem cell genes we observed, particularly Krt15, suggests a lineage related, distinct cell population that has increased stemness and is more prevalent in NAMPT treated wounds versus control wounds.

### Pathway Analysis Reveals Effect of NAMPT on Diabetic Wounds

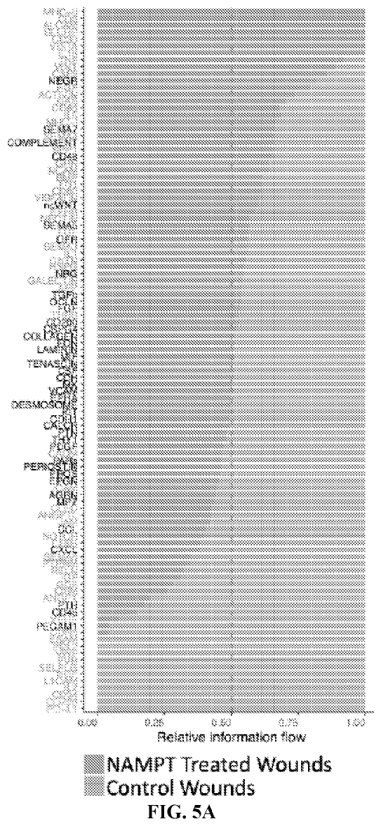

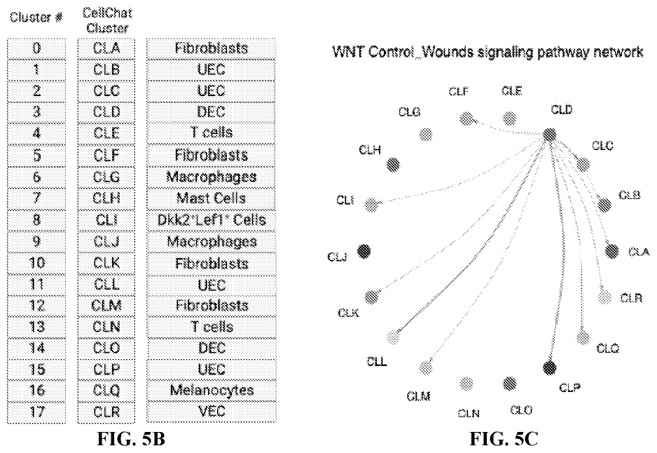

We utilized the CellChat package to infer cell-cell communication in NAMPT treated diabetic wounds versus control diabetic wounds. FIG. 5A shows that NAMPT treatment promoted several pathways that play critical roles in the function of epidermal stem cells including Wnt (FIG. 5A-D), bone morphogenetic protein (Bmp) (data not shown), and Activin (data not shown).

Wnt signaling involves binding of secreted Wnt ligands binding to Frizzled receptors, leading to the activation of Disheveled and stabilization of β-catenin, which translocates into the nucleus to activate transcription. Wnt signaling plays critical roles in skin development¹⁵. Wnt signaling is required for homeostasis of hair follicles and the interfollicular epidermis through modulation of stem cell populations^{16,17}. (Cellchat clusters are named CLA-CLR, which correspond to clusters #0-#17, respectively; FIG. 5B). As determined by CellChat analysis, NAMPT treatment increased diversity, number, and strength in the Wnt-Fzd ligand-receptor interactions (FIG. 5A-D). In particular, increased Wnt signaling was observed among undifferentiated and differentiated epidermal cell populations, suggesting activation of these cell populations toward enhanced wound healing and hair follicle regeneration. Activins are secreted ligands that mediate their biological effects though binding of transmembrane receptor serine/threonine activin receptors. Activins are essential to skin morphogenesis, hair follicle development, and wound healing¹⁸⁻²⁰. Bone morphogenic proteins (BMPs) are ligands that bind cell surface serine/threonine kinase receptors and activate Smad proteins that translocate into the nucleus and influence transcription²¹. BMP signaling is also involved in regulation of epidermal and hair follicle stem cells²².

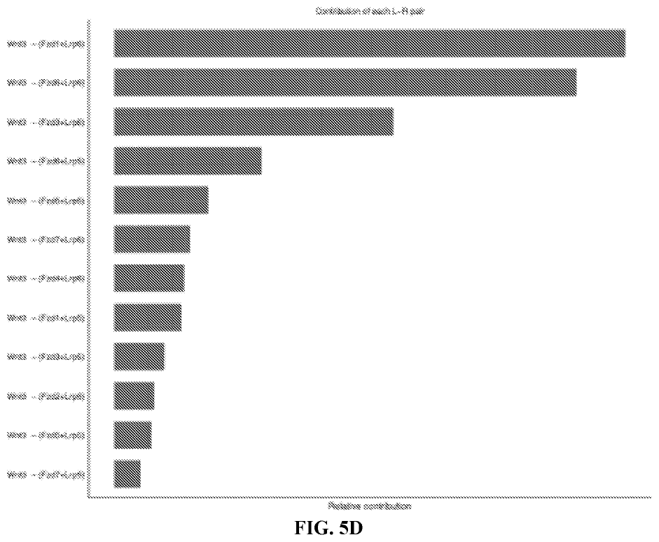

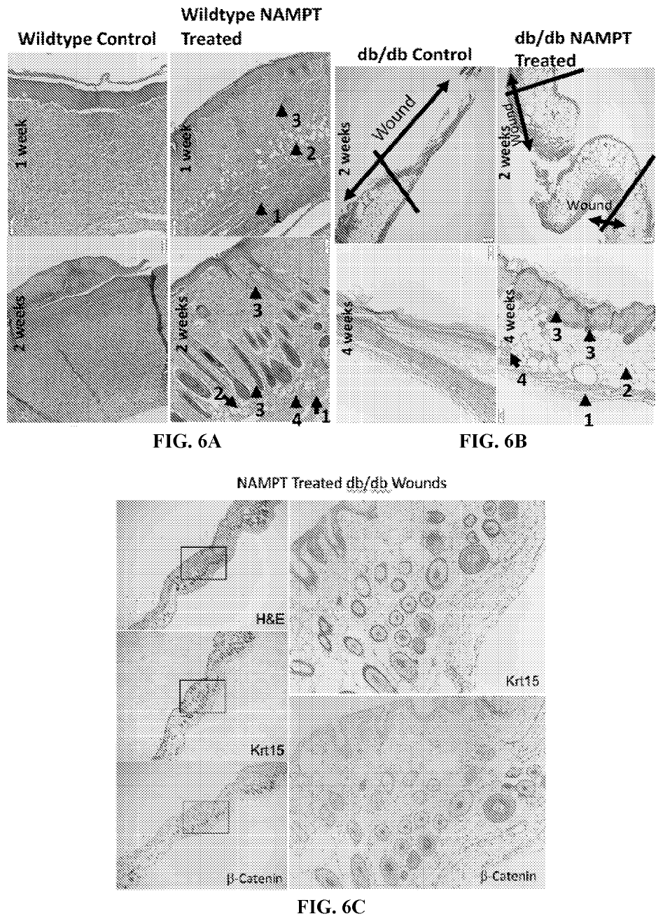

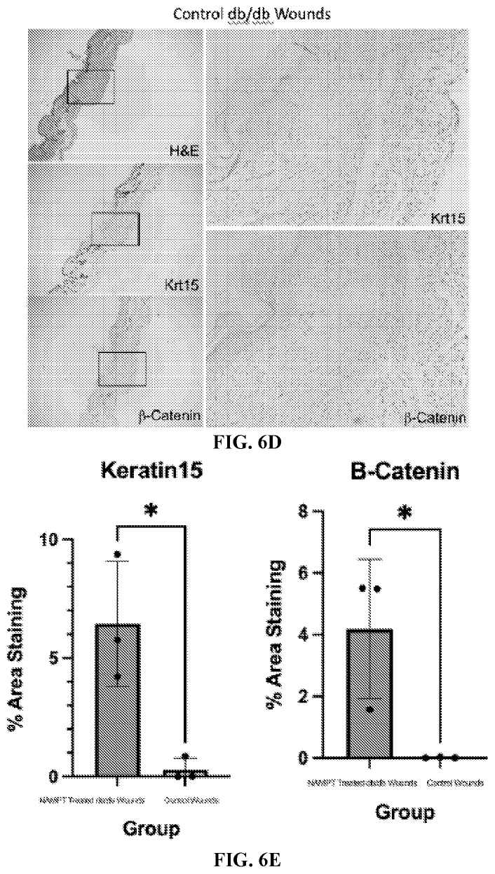

The increased Activin, Bmp, and Wnt¹⁶ activation, particularly among epidermal cell populations upon NAMPT treatment, may suggest increased regenerative signaling, such as in hair follicle formation, confirming our histological findings (FIG. 6). For example, Wnt and BMP signaling crosstalk is critical in the development of hair²³.

Epidermal stem cells express high levels of Itgb1^{24,25}. Itgb1 positive epidermal cells demonstrate multipotent differentiation capacity, and depend on Itgb1 for these functions²⁵. Hgf accelerates wound healing by promoting dedifferentiation (increasing stemness) of epidermal cells through Itgb1²⁶. Our data demonstrate that NAMPT promoted Hgf signaling in epidermal stem cell populations, which also express higher levels of Itgb1 because of NAMPT treatment (FIG. 3). Hgf deficiency in diabetic mesenchymal stem cells (MSCs) may underly their decreased efficacy in wound healing²⁷; it is, therefore, possible that NAMPT-mediated restoration of Hgf signaling contributed to the ability of NAMPT to promote wound healing.

We also observed that immune pathways were upregulated in NAMPT treated diabetic wounds. For example, MHC-II, CD6, ALCAM, VISTA, MHC-I, and CD80 were enriched in NAMPT treated diabetic wounds. This may suggest a priming of the wound environment against infection through enhanced antigen presentation and immune system activation.

Additionally, we observed the activation of several pro-inflammatory pathways, including TNF and IL1 in NAMPT treated diabetic wounds. TNF and IL1 are critical in promoting epidermal cell proliferation and migration in wound healing. These findings are also supported by the recent finding in humans that diabetic foot ulcers that heal demonstrate higher pro-inflammatory signaling, compared to diabetic foot ulcers that do not heal. NAMPT also increased progranulin (Grn) signaling (FIG. 5). Grn has been shown to attract immune cells to the wound site and stimulate angiogenesis and cell proliferation, suggesting its growth factor properties. These findings suggest that NAMPT treatment may stimulate the immune system which may prime the diabetic wound microenvironment against infection.

In addition, we observed an increased activation of Colony Stimulating Factor (CSF3), which is also known as granulocyte-macrophage colony stimulating factor (GM-CSF) (data not shown). Specifically, Cluster 5 (CLF) fibroblasts in the NAMPT treated wounds signal CSF3 to Cluster 6 (CLG) macrophages, likely enhancing CLG activation. Interestingly, we observed increased SPP1 signaling emerging from NAMPT treated CLG macrophages and targeting multiple cell populations, predominantly epidermal cell populations (data not shown). Osteopontin (SPP1) has been shown to activate mesenchymal stem cells to improve wound healing. Osteopontin deficient mice display altered wound healing with disorganization of the extracellular matrix including small diameter collagen fibrils.

### Pseudotime Lineage Trajectory Analysis Reveals NAMPT Stimulated Hair Follicle Formation Through Stimulation of Keratin 79+ Cells.

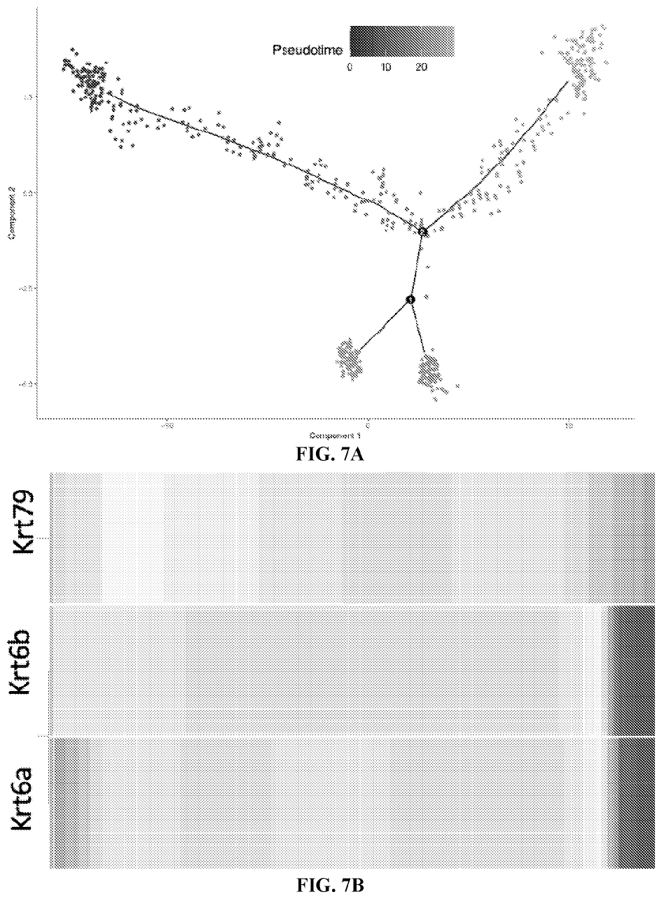

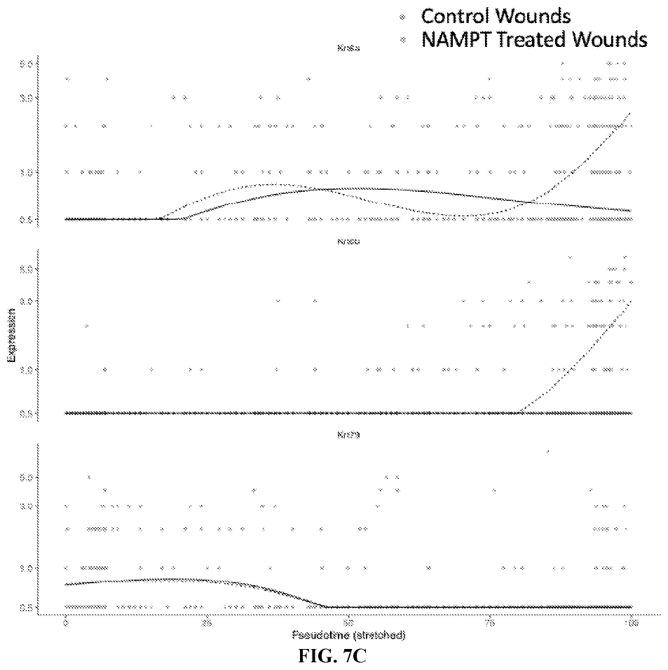

It was previously demonstrated that Keratin79+ (Krt79+) epidermal cells migrate during the formation of a new hair canal. Next, during the early stages of differentiation to hair follicle, Krt79+ cells act as early progenitor cells for the companion layer. These cells then become Krt79-− and Keratin6+ (Krt6+). Here, we perform lineage tracing to identify this trend in diabetic wounds. Cells were ordered by pseudotime; Krt79+ cells appeared early, representing progenitor cells, and Krt6a+ and Krt6b+ cells appeared late, representing differentiated cells. This confirmed that the pseudotemporal directionality was biologically accurate (FIG. 7A-C). FIG. 7C demonstrates that Krt79+ and Krt6a/Krt6b+ cells are enriched in NAMPT treated wounds relative to control treated wounds. This suggests that this process of Krt79+ to Krt6+ differentiation is induced by NAMPT treatment, and contributes to hair follicle formation, which is consistent and confirmed by our histological findings (FIG. 6).

### Pseudotime Lineage Trajectory Analysis Reveals NAMPT Treatment Stimulated Sebaceous Gland Formation

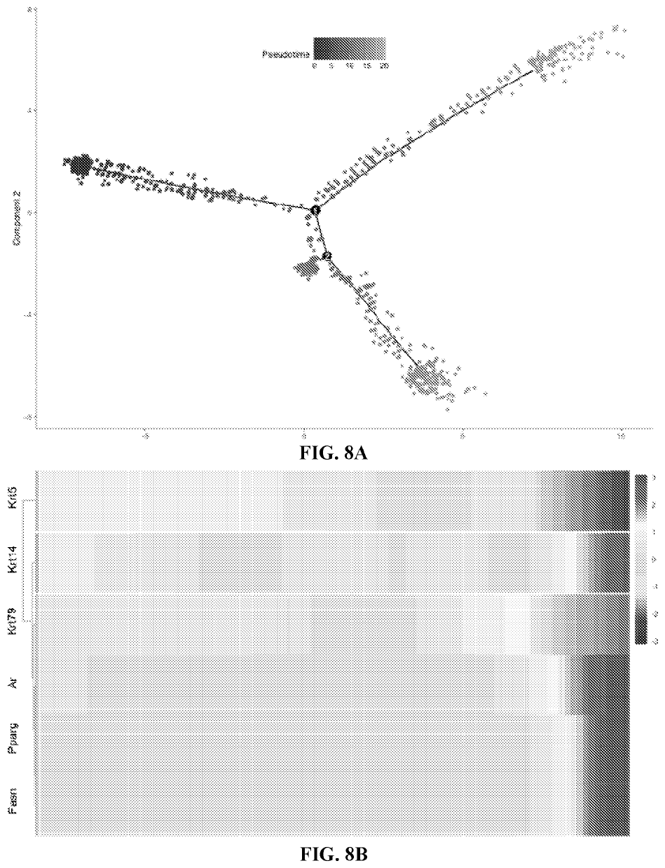

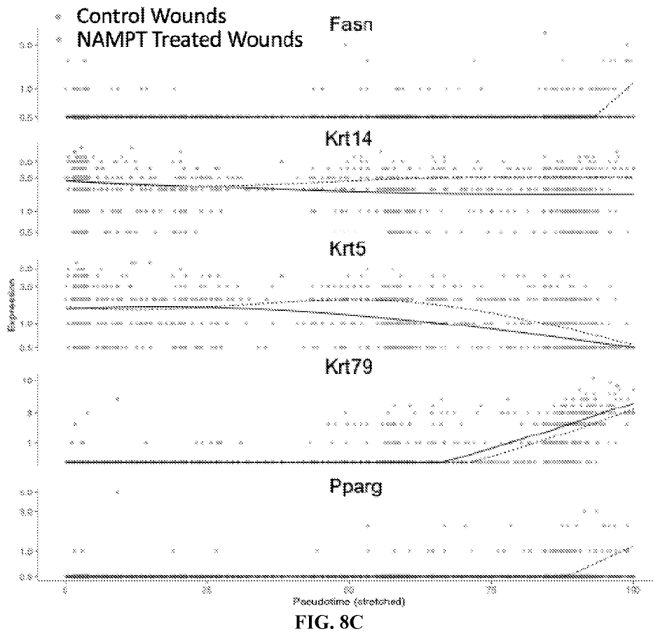

Krt5+/Krt14+ positive epidermal cells are found in the basal tip of the sebaceous gland and represent progenitor/stem cells that have potential to differentiate into sebocytes37. During early differentiation, Krt5+/Krt14+ cells lose Krt5, retain Krt14, and gain Krt79 37. Later during differentiation, these Krt14+/Krt79+ cells become Pparg+/Ar+/Fasn+ (Pparg, Peroxisome proliferator-activated receptor gamma; Ar, androgen receptor; Fasn, fatty acid synthase)37. Here, we performed lineage tracing to identify this trend in diabetic wounds. Cells were ordered by pseudotime; Krt5+/Krt14+ cells appeared early, representing stem/progenitor cells, and Pparg+/Ar+/Fasn+ appeared late, representing differentiated sebocytes (FIG. 8A-C). These results confirmed that the pseudotemporal directionality was biologically accurate relative to the known differentiation program of cell progenitor cells differentiating into sebocytes. Results shown in FIG. 8C demonstrated that Pparg+/Fasn+ cells are enriched in NAMPT treated wounds relative to control wounds. This suggests that the Krt5+/Krt14+ cell to Pparg+/Fasn+ differentiation axis is induced by NAMPT treatment, and largely absent in control wounds, and contributes to sebaceous gland formation, which is consistent and confirmed by our histological findings (FIG. 6).

### Pseudotime Lineage Trajectory Analysis Reveals NAMPT Stimulates a Krt77+ Cell Population

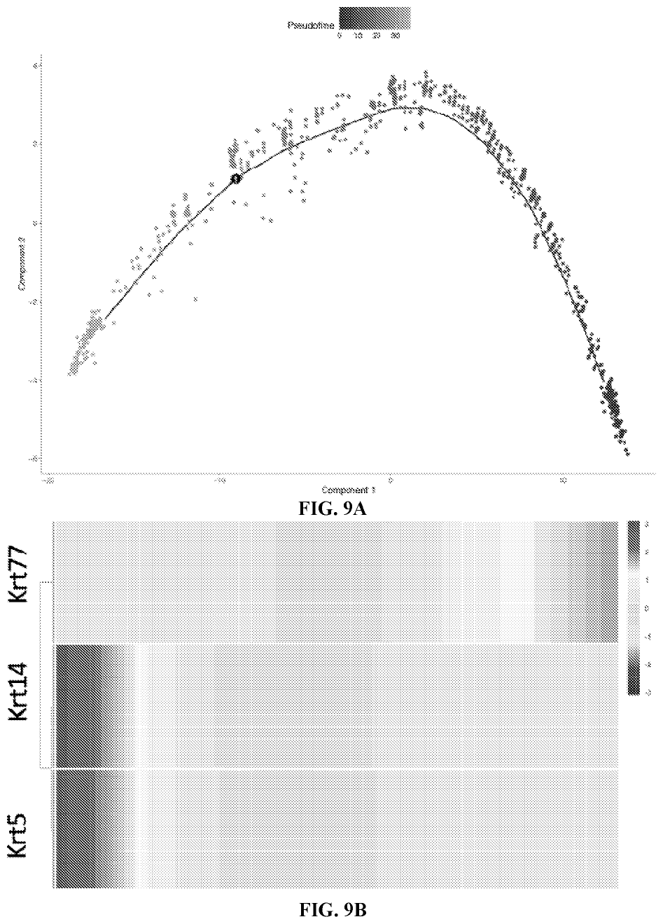

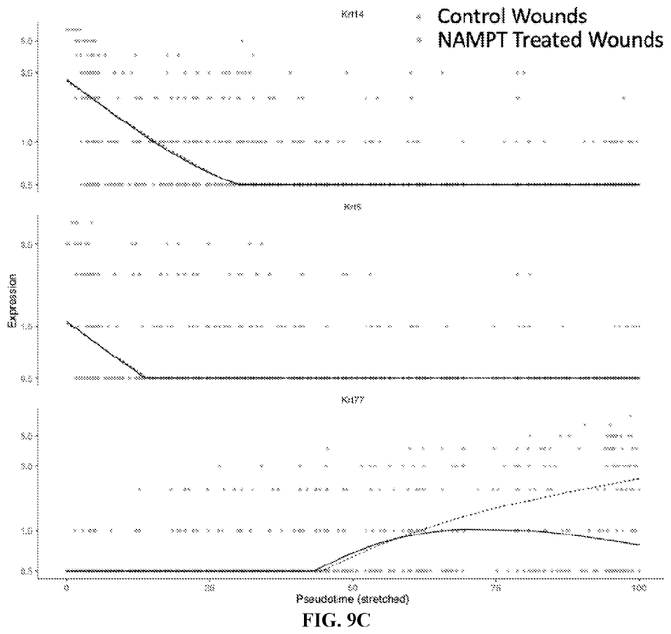

In the mouse, sweat glands are present only in the paw pads38, which prevents the study of sweat glands and their regeneration in the dorsal skin. Krt77 is a marker of eccrine sweat glands39. Krt5+/Krt14+ progenitor cells differentiate into Krt77+ sweat gland duct cells39. Here, we performed lineage tracing to identify this differentiation pattern of sweat gland cells in diabetic wounds. Cells were ordered by pseudotime; Krt5+/Krt14+ cells appeared early, representing stem/progenitor cells, and Krt77+ appeared late, representing sweat gland cells (FIG. 9A-C). This analysis confirmed that the pseudotemporal directionality was biologically accurate, differentiating towards Krt77+ cells. FIG. S4C demonstrates that Krt77+ cells are increased in NAMPT treated wounds, relative to control wounds. This suggests that the differentiation process of Krt5+/Krt14+ to Krt77+ cell differentiation is induced by NAMPT treatment, and largely absent in control wounds. This molecular evidence suggests that NAMPT may promote differentiation to a Krt77+ cell population, that may possibly have some relation to sweat glands.

### Role of NAMPPT in Epidermal Cell Differentiation

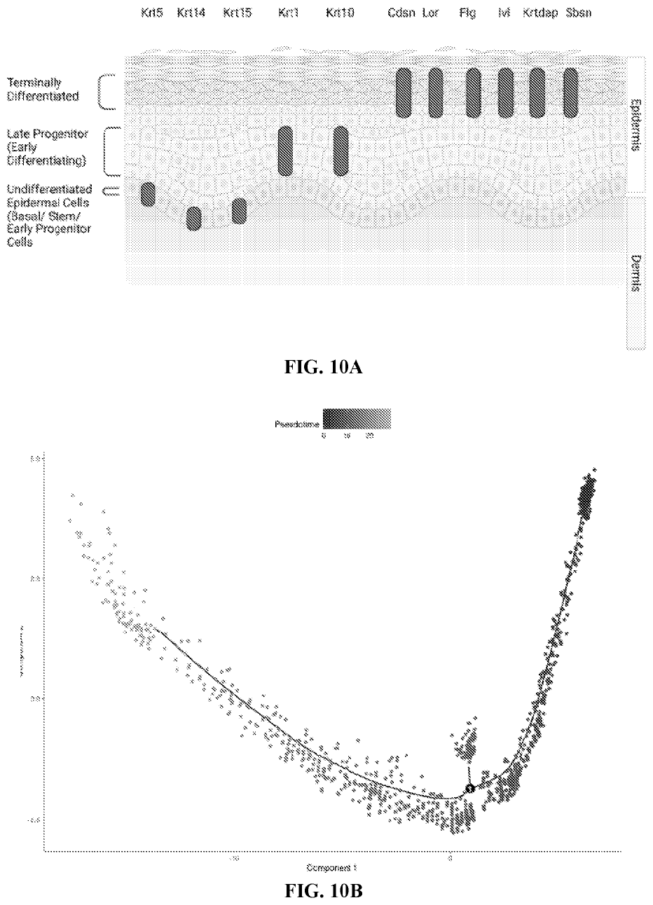

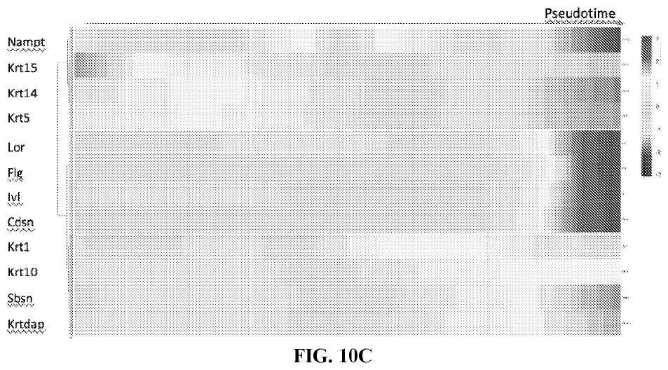

During epidermal cell differentiation, undifferentiated basal cells, characterized by the Krt5+/Krt14+/Krt15+ signature, differentiate into Krt1+/Krt10+ spinous cells, which finally differentiate into terminally differentiated keratinocytes (Cdsn+/Lor+/Ivl+) (FIG. 10A). To investigate the role of endogenous NAMPT in epidermal differentiation, we plotted all epidermal cells from nondiabetic skin as a function of pseudotime. Undifferentiated epidermal cells appeared at the beginning of the trajectory at early pseudotime. Differentiated epidermal cells appeared at the end of the trajectory at late pseudotime. We confirmed that the plotted trajectory and pseudotemporal directionality was biologically accurate by plotting several markers as a function of pseudotime (FIGS. 10B and C). As expected, Krt5, Krt14, and Krt15 were highly expressed at early pseudotime in undifferentiated epidermal cells. As Krt5, Krt14, and Krt15 decreased, Krt1 and Krt10 increased, as expected for differentiating epidermal cells. Finally, terminal differentiation markers, including Cdsn, Lor, Ivl, and Sbsn, were enriched in differentiated cells at the end of the trajectory (late pseudotime). We observed high NAMPT levels in early epidermal stem cells expressing high levels of Krt5, Krt14, and Krt15. The pseudotemporal decrease in NAMPT preceded the decreases in stem markers, suggesting NAMPT is associated with maintenance of stemness. Interestingly, NAMPT expression increased prior to the increase of Krt1 and Krt10, markers of more differentiated epidermal cells, suggesting NAMPT is also associated with commitment to differentiation. NAMPT expression is completely lost in terminally differentiated epidermal cells, which have no intrinsic sternness (FIG. 10C).

### Cell Cycle Analysis Reveals Effect of NAMPT on Diabetic Wounds Cell Proliferation

Impaired cellular proliferation in diabetic wounds leads to delayed granulation tissue formation and delayed reepithelialization. We utilized the Tricycle package40 to determine the impact of NAMPT on cell cycle of diabetic wounds. NAMPT treatment increases the proportion of cells in the S phase, G2/M phase, and M phase compared to controls, suggesting enhanced cellular proliferation (data not shown). We confirmed our finding by running KEGG pathway analysis, which revealed that NAMPT treatment significantly enhances the “KEGG_CELL_CYCLE” pathway (data not shown). These findings were confirmed by running HALLMARK pathway analysis, which revealed that NAMPT treatment significantly enhances the “HALLMARK_E2F_TARGETS” and “HALLMARK_MITOTIC_SPINDLE” pathways (data not shown).

### Pathway Analysis Reveals Effect of NAMPT on Diabetic Wound Metabolism

We utilized KEGG pathway analysis, which revealed that NAMPT treatment significantly enhances the “KEGG_OXIDATIVE_PHOSPHORYLATION”, “KEGG_FATTY_ACID_METABOLISM”, “KEGG_CITRATE_CYCLE_TCA_CYCLE”, “KEGG_MTOR_SIGNALING”, and “KEGG_PYRUVATE_METABOLISM” pathways (data not shown). We further confirmed our findings using HALLMARK pathway analysis, which revealed that NAMPT treatment significantly enhances the “HALLMARK_OXIDATIVE_PHOSPHORYLATION” pathway (data not shown). These findings suggest that NAMPT increases metabolic activity of diabetic wound tissues.

### NAMPPT Improves Skin Regeneration

To confirm our findings histologically we showed that application of NAMPT-hydrogel enhanced skin regeneration compared to control vehicle hydrogel in both diabetic and non-diabetic mouse wounds. Tissue analysis indicated that NAMPT-hydrogel treatment promotes skin regeneration including regeneration of hair follicles, sebaceous glands, adipose tissue, the muscular panniculus carnosus and non-scarring patterns of collagen (FIG. 6). NAMPT-hydrogel also promotes robust angiogenesis in both non-diabetic and diabetic mouse wounds, promoting regeneration of the newly forming skin. Immunohistochemistry revealed significantly increased Keratin15 and B-catenin in NAMPT treated diabetic wounds, compared with control diabetic wounds, which is consistent with our scRNAseq findings (FIG. 6D-F). The increased B-catenin suggests increased Wnt signaling activation. These findings confirm our scRNAseq findings, including increased epidermal stem cell populations and activity, as well as lineage analyses suggesting enhanced sebaceous gland and hair follicle formation. We observed basket weave collagen deposition and lack of scarring in the NAMPT treated wounds, which is confirmed by increased activity of wound responsive fibroblasts and regenerative signaling such as Wnt, as demonstrated by scRNAseq.

As shown above, NAMPT treatment restored normal skin characteristics including sebaceous glands (SG) and hair follicles (HF) without scarring. Single-cell analysis revealed that NAMPT increased Krt15, Itga6, and Itgb1 epidermal stem cell (ESC) populations and promoted regenerative function of ESCs and fibroblasts in wounds. Lineage-inference analysis confirmed that NAMPT promoted ESCs, SG, and HF cell populations. Mechanistically, NAMPT promoted Wnt, Hgf, Csf3, and Fn1-signaling, cell migration, proliferation, metabolism, immune function, and normalization toward nondiabetic state. Immunohistochemistry confirmed increased Krt15 cell populations and Wnt signaling through increased β-catenin in NAMPT treated wounds. These suggest therapeutic potential of NAMPT to promote skin regeneration from ESCs in diabetic and nondiabetic states.

Those skilled in the art will recognize, or be able to ascertain using no more than routine experimentation, many equivalents to the specific embodiments in accordance with the present disclosure. The scope of the present disclosure is not intended to be limited to the above Description, but rather is as set forth in the appended claims.

What is claimed is:

1. A method for stimulating organ regeneration, including hair growth and/or regrowth and/or wound healing in a subject, the method comprising contacting a target region of skin of the subject with an effective amount of a hair growth or regrowth composition comprising a nicotinamide phosphorylribosyltransferase (NAMPT) protein, an active peptide fragment thereof, a NAMPT analog, a vector comprising a polynucleotide encoding a NAMPT protein, a NAMPT activator, or a combination thereof.

2. The method of claim 1, wherein the composition comprises a NAMPT protein according to SEQ ID NO: 1.

3. The method of claim 1, wherein the composition comprises a NAMPT analog that is at least 70% identical to SEQ ID NO: 1.

4. The method of claim 1, wherein the composition comprises a NAMPT activator, wherein the NAMPT activator is 3,6-dibromo-α-[(phenylamino)methyl]-9H-carbazol-9-ethanol (P7C3), 1-[4-(8-Oxa-3-azabicyclo[3.2.1]octane-3-sulfonyl)-phenyl]-3-pyridin-4-ylmethylurea (SBI-797812), or combinations thereof.

5. (canceled)

6. The method of claim 1, wherein the composition comprises both the NAMPT protein and the NAMPT activator.

7. The method of claim 1, wherein the composition comprises a vector comprising RNA encoding a NAMPT protein, and wherein the NAMPT protein is according to SEQ ID NO: 1.

8. The method of claim 1, wherein the composition further comprises an NAD precursor, and wherein the NAD precursor is selected from the group consisting of: tryptophan, nicotinic acid (pyridine-3-carboxylic acid), nicotinamide (nicotinic acid amide), nicotinamide mononucleotide (NMN) and nicotinamide riboside (NR).

9. The method of claim 1, wherein the composition further comprises a pharmaceutically acceptable carrier, wherein the pharmaceutically acceptable carrier comprises a topical formulation or a hydrogel, and wherein the hydrogel is selected from the group consisting of Pluronic-F127 hydrogel, fibrin hydrogels, carboxymethyl cellulose hydrogels, PEGs, polysaccharides, and combinations thereof.

10. (canceled)

11. (canceled)

12. The method of claim 1, wherein the target region of skin is alopecia-affected skin, and wherein the alopecia-affected skin is part or all of the scalp of the subject.

13. (canceled)

14. The method of claim 1, further comprising the step of facilitating penetration of the skin of the target region using a skin penetration enhancer selected from needles, abrasive materials, or the application of high pressure to the skin.

15. The method of claim 1, further comprising the step of creating a border around the target region to restrict activity of the NAMPT to substantially within the target region.

16. A wound healing composition for topical use, comprising a dosage of nicotinamide phosphorylribosyltransferase (NAMPT) protein, an active peptide fragment thereof, or a NAMPT analog, and a pharmaceutically acceptable topical carrier, wherein administration of the composition on the skin of a patient promotes complete skin regeneration.

17. The wound healing composition of claim 16, wherein the topical carrier is a hydrogel, wherein the hydrogel is selected from the group consisting of Pluronic-F127 hydrogel, fibrin hydrogels, carboxymethyl cellulose hydrogels, PEGs, polysaccharides, and combinations thereof.

18. The wound healing composition of claim 16, wherein the composition further comprises a NAMPT activator, wherein the NAMPT activator is 3,6-dibromo-α-[(phenylamino)methyl]-9H-carbazol-9-ethanol (P7C3), 1-[4-(8-Oxa-3-azabicyclo[3.2.1]octane-3-sulfonyl)-phenyl]-3-pyridin-4-ylmethylurea (SBI-797812), or combinations thereof.

19. (canceled)

20. The wound healing composition of claim 16, wherein the composition further comprises a NAD precursor, and wherein the NAD precursor is selected from the group consisting of: tryptophan, nicotinic acid (pyridine-3-carboxylic acid), nicotinamide (nicotinic acid amide), nicotinamide mononucleotide (NMN) and nicotinamide riboside (NR).

21. A method of treating a skin wound, comprising administering a therapeutically effective amount of the composition of claim 16 to the wound of a human or animal subject.

22. The method of claim 21, wherein the pharmaceutically acceptable topical carrier is a hydrogel.

23. The method of claim 21, wherein the skin wound is a diabetic wound.

24. The method of claim 21, wherein the wound healing composition comprises at least 0.01 micrograms of the NAMPT protein for every 1 mm2 of skin wound surface area.

25. The method of claim 21, wherein the wound healing composition comprises at least 0.05 micrograms (μg) of the NAMPT protein for every 1 mm2 of skin wound surface area.


SEQUENCE LISTING (1 sequence)

SEQ ID NO: 1 - protein, 491 aa, Homo sapiens [sequence omitted]